(19) United States
(12) Patent Application Publication
(10) Pub. No.: US 2004/0199202 A1
(43) Pub. Date: Oct. 7, 2004

(54) Biological passageway occlusion removal

(75) Inventors: William Dubrul, Belmont, CA (US); Richard Eustis Fulton III, Grand Junction, CO (US)

Correspondence Address: HAYNES BEFFEL & WOLFELD LLP, P O BOX 366, HALF MOON BAY, CA, 94019, US

(73) Assignee: Genesis Technologies LLC, Belmont, CA (US)

(21) Appl. No.: 10/747,813

(22) Filed: Dec. 29, 2003

Related U.S. Application Data
(63) Continuation-in-part of application No. 09/819,350, filed on Mar. 28, 2001, now Pat. No. 6,699,260.
Application No. 09/819,350 is a continuation of application No. 09/189,574, filed on Nov. 11, 1998, now Pat. No. 6,238,412.
(63) Continuation-in-part of application No. 10/463,026, filed on Jun. 17, 2003.
Application No. 10/463,026 is a continuation of application No. 09/844,661, filed on Apr. 27, 2001, now Pat. No. 6,602,204.
Application No. 10/463,026 is a continuation-in-part of application No. 09/588,278, filed on Jun. 5, 2000, now Pat. No. 6,530,923.
Application No. 10/463,026 is a continuation-in-part of application No. 09/336,360, filed on Jun. 18, 1999, now Pat. No. 6,270,464.
Application No. 09/588,278 is a continuation-in-part of application No. 09/248,088, filed on Feb. 9, 1999, now Pat. No. 6,221,006.
(63) Continuation-in-part of application No. 10/457,595, filed on Jun. 9, 2003.
Application No. 10/457,595 is a division of application No. 09/248,083, filed on Feb. 9, 1999, now Pat. No. 6,635,068.
(60) Provisional application No. 60/065,118, filed on Nov. 12, 1997.
(60) Provisional application No. 60/200,546, filed on Apr. 27, 2000.
(60) Provisional application No. 60/137,775, filed on Jun. 4, 1999.
(60) Provisional application No. 60/146,892, filed on Aug. 2, 1999.
(60) Provisional application No. 60/200,546, filed on Apr. 27, 2000.
(60) Provisional application No. 60/154,394, filed on Sep. 17, 1999.
(60) Provisional application No. 60/090,243, filed on Jun. 22, 1998.
(60) Provisional application No. 60/092,734, filed on Jul. 14, 1998.
(60) Provisional application No. 60/114,863, filed on Jan. 6, 1999.
(60) Provisional application No. 60/117,421, filed on Jan. 27, 1999.
(60) Provisional application No. 60/074,199, filed on Feb. 10, 1998.
(60) Provisional application No. 60/105,284, filed on Oct. 22, 1998.
(60) Provisional application No. 60/074,183, filed on Feb. 10, 1998.
(60) Provisional application No. 60/077,281, filed on Mar. 9, 1998.
(60) Provisional application No. 60/104,922, filed on Oct. 20, 1998.

(51) Int. Cl. A61M 29/00

(52) U.S. Cl. 606/200; 606/191; 606/194; 604/96.01; 604/101.01

(57) ABSTRACT

A medical device comprises a catheter having proximal and distal catheter ends and a lumen. The medical device may comprise an expandable and contractible, annular-space-blocking element, carried by the catheter at or near the distal catheter end, having, when in an expanded state, a funnel-shaped surface for receipt of material. The medical device may also comprise first and second expandable and contractible elements. The first element may be a vessel-occluding element positioned distal of the distal catheter end. The second element may be an annular-space-blocking element positioned between the first element and the proximal catheter end. At least one of the elements may comprise spaced apart structural members and a membrane associated therewith. One of the elements, when in an expanded state, may have a funnel-shaped surface and a longitudinally-extending opening for receipt of material.

### CROSS-REFERENCE TO RELATED APPLICATIONS

This application is a continuation-in-part of:

1) Tissue Removal Device and Method, application Ser. No. 09/819,350 Filed 28 Mar. 2001, which is a continuation of Biological Passageway Occlusion Removal, application Ser. No. 09/189,547 Filed 11 Nov. 1998, now U.S. Pat. No. 6,238,412 issued May 29, 2001, which claims the priority of U.S. Provisional Application Ser. No. 60/065,118, filed on Nov. 12, 1997;

2) Intraoperative Tissue Treatment Methods, U.S. patent application Ser. No. 10/463,026 Filed 17 Jun. 2003, which is a continuation of application Ser. No. 09/844,661 filed 27 Apr. 2001 entitled Intraoperative Tissue Treatment Methods, which claims the benefit of Provisional Application No. 60/200,546 filed Apr. 27, 2000 and entitled Diagnostic And Therapeutic Apparatuses And Methods For Use.

Application Ser. No. 09/844,661 is a continuation-in-part of the U.S. patent application Ser. No. 09/588,278 filed Jun. 5, 2000. Application Ser. No. 09/588,278 claims the benefit of the following provisional patent applications: Provisional Application No. 60/137,775 filed Jun. 4, 1999 and entitled TISSUE REMOVAL APPARATUS AND METHOD FOR USE; Provisional Application No. 60/146,892 filed Aug. 2, 1999 entitled DISEASE PREVENTING SHEATH APPARATUS AND METHODS FOR USE; Provisional Application No. 60/200,546 filed Apr. 27, 2000 and entitled DIAGNOSTIC AND THERAPEUTIC APPARATUSES AND METHODS FOR USE; Provisional Application No. 60/154,394 filed Sep. 17, 1999 and entitled ONCOLOGICAL APPARATUS AND METHOD FOR USE. Application Ser. No. 09/588,278 is also a continuation-in-part of U.S. patent application Ser. No. 09/336,360 filed Jun. 18, 1999 entitled BIOPSY LOCALIZATION METHOD AND DEVICE, which application claims priority from the following provisional applications:

a. Application No. 60/090,243, filed Jun. 22, 1998;

b. Application No. 60/092,734, filed Jul. 14, 1998;

c. Application No. 60/114,863, filed Jan. 6, 1999; and

d. Application No. 60/117,421, filed Jan. 25, 1999.

Application Ser. No. 09/588,278 is also a continuation-in-part of U.S. patent application Ser. No. 09/248,088 filed Feb. 9, 1999, which application claims benefit of the following provisional applications:

a. Application No. 60/074,199 filed Feb. 10, 1998; and

b. Application No. 60/105,284 filed Oct. 22, 1998; and

3) Occlusion, Anchoring, Tensioning and Flow Direction Apparatus and Methods For Use, U.S. application Ser. No. 10/457,595 Filed 9 Jun. 2003, which is a division of U.S. patent application Ser. No. 09/248, 083 filed Feb. 9, 1999, which claims the benefit of the following provisional patent applications: 60/074,183 filed Feb. 10, 1998, 60/077,281 filed Mar. 9, 1998, 60/104,922 filed Oct. 20, 1998.

### BACKGROUND OF THE INVENTION

The present invention relates to medical devices and methods.

One aspect of this invention relates to a removal device for a biological occlusion and more particularly to a catheter and occlusion engaging element which is adapted to the removal of blockages in hemodialysis grafts. There are many techniques and devices known in the art for removing blockages in the vascular system and other passageways of the human body.

Another aspect of, the present invention is directed to procedures, including biopsy and tumorectomy methods, and associated apparatus which provide for less invasive techniques while also providing for enhanced tissue specimens being retrieved.

Another aspect of the present invention relates to improved guide wires or catheters and method for their use, where the devices have a distal mechanism that acts as a mechanism for: 1. Flow Directed, using the natural flowing fluids, pressure differentials or contractile forces of the body onto the distal mechanism to direct its motion and direction or 2. Anchored, so that once the device is in the desired location, it can be anchored against the tissue where it rests; 3. Tensioned, so that placement of a device, over the guide wire is accomplished with less difficulty and 4. Occluded, so that vessels and aneurysms can be occluded.

Guide wire management in the operating room is problematic, and threading the needle of the arteries or other vessels including, but not limited to veins, intestines, fallopian tubes, etc. to reach the area to be treated is difficult. Further, once the guide is in the desired location, it is often difficult to make certain that the it remains in that location. Even further, once the guide wire, catheter, endoscope or other device is in the desired location and another device is placed over, through or along side it, the initially placed device has a tendency to move due to the forces exerted on it when other devices are using it as a guide.

Additionally, other anchors are required for attaching tissue or other matter to improved or different locations within the body.

Even further, vessel occluders are often required for a variety of medical procedures.

For these reasons, it is desirable to provide an improved devices and methods for their use, which facilitate 1. using the physiologic motions of the body to help direct the device. In addition, flow pressure differential can be artificially created or enhanced by the technician/physician so that this same technology can be used when physiologic means is unavailable or insufficient. Further, the natural contractile forces of the body (e.g. those of the intestinal tract, gall bladder, esophagus, etc.) can be harnessed so that the device including, but not limited to guide wires, catheters, endoscopes, etc. are moved along with those forces. 2. Even further, it is desirable to provide a device that has an anchoring mechanism on it so that it will not move once in its desired position. 3. And yet even another desired characteristic would be to provide an anchored device that has a tensioning characteristic applied to it for placement of other devices over through or along side the first placed device. 4. And finally, another desired characteristic is that of a simple and effective occlusion system.

There is a continuing need for improved devices to meet at least the following objectives.

The first objective is to reduce cost. This is particularly important in recent years where it is clear for safety and sanitary reasons that these will be single use devices. A device, even though it performs a function in some improved manner, will not be widely used if it is considerably more costly than the alternatives available.

A second objective is to provide a device that is simple to use and in a very real sense simple to understand. This will encourage its adoption and use by medical personnel. It will also tend to keep cost low.

The third objective is to provide a device that entails a procedure with which the medical profession is familiar so that the skills that have been learned from previous experience will continue to have applicability.

A fourth objective relates to the effectiveness and thoroughness with which the blockage is removed. It is important that a maim amount of the blockage be removed; recognizing that no device is likely to provide one-hundred percent removal.

A fifth objective concerns safety; a matter which is often so critical as to trump the other considerations. It is important to avoid tissue trauma. In many circumstances, it is critically important to avoid breaking up a blockage in a fashion that leads to flushing elements of the blockage throughout the body involved.

There are trade-offs in design considerations to achieve the above five interrelated objectives. Extreme simplicity and a very simple procedure might over compromise safety. Addressing all of these considerations calls for some trade-off between the objectives.

Accordingly, an object of this invention is to provide an improved removal device for a body passageway blockage which achieves the objectives of reduced cost, enhanced simplicity, a standard procedure, high effectiveness and a high degree of safety. Most particularly, it is an object of this invention to achieve these objectives with an enhanced trade-off value for the combined objectives.

Another object of this invention is to provide an improved occlusion, tensioning, anchoring and flow device that achieves the objectives of reduced cost, enhanced simplicity, a standard procedure, high effectiveness and a high degree of safety. Most particularly, it is an object of this invention to achieve these objectives with an enhanced trade-off value for the combined objectives.

For these reasons, it is desirable to provide an improved device that may circumvent some of the problems associated with previous techniques. This improved medical device provides a new configuration that will eliminate some of those problems and methods for their use, which facilitate removal of vascular obstructions in the operating room or interventional suite.

### SUMMARY OF THE INVENTION

A first aspect of the invention is directed to a medical device comprising a catheter having a proximal catheter end and a distal catheter end and defining a lumen extending from the distal catheter end towards the proximal catheter end. The medical device also comprises first and second expandable and contractible elements. The first expandable and contractible element is a vessel-occluding element positioned distal of the distal catheter end. The second expandable and contractible element is an annular-space-blocking element positioned between the first expandable and contractible element and the proximal catheter end. At least one of the first and second expandable and contractible elements comprises spaced apart structural members and a membrane associated therewith.

A second aspect of the invention is directed to a medical device comprising a catheter having a proximal catheter end and a distal catheter end and defining a lumen extending from the distal catheter end towards the proximal catheter end. The medical device also comprises first and second expandable and contractible elements. The first expandable and contractible element is a vessel-occluding element positioned distal of the distal catheter end. The second expandable and contractible element is an annular-space-blocking element positioned between the first expandable and contractible element and the proximal catheter end. A chosen one of the first and second expandable and contractible elements, when in an expanded state, has a funnel-shaped surface and a longitudinally-extending opening to permit material to pass therethrough for receipt of material.

A third aspect of the invention is directed to a medical device comprising catheter having a proximal catheter end and a distal catheter end and defining a lumen extending from the distal catheter end towards the proximal catheter end. The medical device also comprises a support element extending distally of the distal catheter end. The medical device further comprises first and second expandable and contractible elements. The first expandable and contractible element is a vessel-occluding element positioned distal of the distal catheter end. The second expandable and contractible element is an annular-space-blocking element positioned between the first expandable and contractible element and the proximal catheter end. A chosen one of the first and second expandable and contractible elements, when in an expanded state, has a funnel-shaped surface and a longitudinally-extending opening to permit material to pass therethrough for receipt of material. At least one of the first and second expandable and contractible elements comprises spaced apart structural members and a membrane associated therewith.

A fourth aspect of the invention is directed to a medical device comprising a catheter having a proximal catheter end and a distal catheter end and defining a lumen extending from the distal catheter end towards the proximal catheter end. The medical device also comprises a first expandable and contractible, vessel-occluding element positioned distal of the distal catheter end and a second expandable and contractible, annular-space-blocking device-occluding element positioned between the first expandable and contractible element and the proximal catheter end.

A fifth aspect of the invention is directed to a medical device comprising a catheter having a proximal catheter end and a distal catheter end and defining a lumen extending from the distal catheter end towards the proximal catheter end. The medical device also comprises an expandable and contractible, annular-space-blocking element carried by the catheter at or near the distal catheter end. The expandable and contractible element, when in an expanded state, has a funnel-shaped surface for receipt of material. The expandable and contractible element also has spaced apart structural members and a membrane associated therewith.

### BRIEF DESCRIPTION

In brief one embodiment of this invention is particularly adapted to the removal of blockages in hemodialysis grafts. That embodiment combines a catheter having a blocking feature that blocks the annulus between the catheter and the graft and a support wire having an occlusion engaging element.

The support wire extends through the catheter, through or around the occlusion and at its distal end has an annular braided element attached thereto. The support wire is a dual element support wire having a core and an annular shell that slides on the core. The distal end of the core is attached to the distal end of the annular braided element and the distal end of the shell is attached to the proximal end of the annular braided element. Thus movement of the core and shell relative to one another moves the braided element from a radially retracted position which is useful for insertion through the catheter to a radially expanded position which expands it to the sidewall of the graft. When the annular braided element is in its radially compressed state, it can be passed through the occlusion together with the rest of the wire to reside on the distal end of the occlusion. When the braided element is expanded and moved proximally (that is, in a retrograde fashion), it will engage the occlusion and force the occlusion into the catheter. Alternatively, no motion of the engaging element may be required if aspiration is applied. In this case, the engaging element acts as a seal to prevent the suction from aspiration to remove much material beyond its point of deployment in the channel.

The distal end of the catheter is proximal of the occlusion and contains a blocking mechanism that extends radially from the distal end of the catheter to the wall of the graft or body passageway. This catheter blocking element also has a radially retracted insertion state and a radially expanded blocking state. The blocking element is a multi-wing malecot type device which is covered by a thin elastomeric film or membrane.

This malecot type of device is bonded to the distal end of the catheter or an integral part of the catheter. The distal tip of the dilator, over which the catheter is inserted, has a slightly increased diameter. This tip is in the nature of a ferrule. When the dilator is removed, the ferrule abuts against the distal end of the multi-wing malecot pushing this blocking element from its radially compressed state into its radially expanded state. Alternatively, the tip of the dilator can be bonded to the catheter with a break-away bond so that when the dilator is removed, the blocking element is expanded in a similar fashion. In this radially expanded state, the malecot and its film cover blocks the annulus around the catheter so that the occluded blood or other obstruction which is being removed is forced into the catheter where it is aspirated or otherwise removed.

Conversely, it is understood that the blocking element could be fabricated from tubular braid and the engaging element could be formed from the malecot style configuration.

Another embodiment of this invention is particularly adapted to the anchoring of wires or tubes within the tubular channels of the body including, but not limited to veins, arteries, intestines, nasal passages, ear canal, etc. Further, this anchoring embodiment has a applicability in applying an anchor to tissues or other matter to areas of the body other than in tubular channels including, but not limited to the face, breast joints, etc. This embodiment has a support wire with an engaging element.

The support wire is a dual element support wire having a core and an annular shell that slides on the core. The distal end of the core is attached to the distal end of the annular braided element and the distal end of the shell is attached to the proximal end of the annular braided element. Thus movement of the core and shell relative to one another moves the braided element from a radially retracted position which is useful for insertion into the body to a radially expanded position which expands it to the sidewall of the tubular channel or against other tissue or matter within the body. When the annular braided element is in its radially compressed (smaller diameter) state, it can be passed through or around occlusions together with the rest of the wire to reside on the distal end of the occlusion in the case of tubular channels with occlusions. It is a preferred embodiment of the instant invention that it can be made very small. When the braided element is expanded and pulled proximally (that is, in a retrograde fashion), it will engage the walls of the tubular channel and the elongate support wire can be put into tension. This distal engaging tubular braid element may or may not be covered by or integrated with a thin film or membrane to create patency or other desirable characteristics.

The instant invention also describes another use of the same device of the instant invention with minor changes. In this case, the tubular braid distal expansile mechanism may be used on the end of a guide wire or catheter so that once deployed in a tubular channel with flow such as arteries and veins, the expanded mechanism can carry the support wire in the direction of the flow. In order to accomplish this flow characteristic of the instant invention, it may be desirable to deploy the distal expanding tubular braid whereby the support wire becomes ‘floppy’ in nature so that it will flow with the expanded ‘umbrella’. The author uses the phrase ‘umbrella’ only as a communication tool in that an umbrella starts out with a small diameter shaft in its un-deployed condition (radially compressed condition) and ends up with a large diameter configuration when deployed. The shape of the expanding mechanism is varied and includes, but is not limited to an umbrella shape, a spheroid shape, an ovoid shape, a conical shape, a disc-shape, etc. The inventors have fabricated at least all of the aforementioned shapes using tubular/annular braid and successfully tested the flow, anchoring, tensioning and occlusion characteristics in both a static and dynamic in vitro environment. Creating the expanded annular braided mechanism is accomplished by pulling the inner wire of the support wire out of the outer tube. The outer tube can be made of very flexible material so that the inner wire gives the structure all of the support. When the ‘umbrella reaches the desired location which is usually determined by image intensification including, but not limited to x-ray, ultrasound, MRI, etc., the inner wire can be re-inserted into the flexible outer tube of the support wire to give the desired support required. Also once the ‘umbrella’ with the flexible outer tube needs to be removed, the inner wire can be an actuator to un-deploy the expanded braided element back to its smaller and radially compressed size. This is accomplished by bonding the outer tube of the support wire to the distal end of the tubular braid expanding element and the inner wire of the support wire is slightly bonded to the distal end of the braided expanding element. This slight bond could also be an interference fit where the inner wire snaps into and out of the distal end of the braided expanding element.

Even further, by making another minor change to the instant invention would be to use the braided expanding element as a permanent or temporary occluder without the support wire being left in place. This is accomplished by having the outer tube not bonded to the proximal end of the expanding element and the inner wire of the support wire to be only slightly bonded to the distal end of the expanding braided element. In this case, the inner wire is pulled in a retrograde direction relative to the outer tube. This action causes the expanding braided element to expand radially. Once the expanding element expands to the desired shape for the particular application and occlusion, the inner wire is pulled out of the ‘snap’ or interference fit on the distal end of the expanding braided element and the expanded braid occluder is left in place when both the inner and outer member of the support wire is removed from the body.

Hence, nearly the same invention allows the use for four different applications in the health care field.

Pertinent descriptions are set forth in a number of issued U.S. patents, including U.S. Pat. Nos. 5,275,611, 5,312,360, 4,696,304, 5,176,659, 5,437,631, 5,606,979, 5,779,672, 5,456,667, 5,733,294 and 5,209,727. A pin vise for helping grip the proximal end of a guide wire is illustrated in U.S. Pat. No. 4,858,810. U.S. Pat. Nos. 5,275,611, 5,312,360 describe a tension guide and dilator. U.S. Pat. No. 5,779,672 describes a detachable inflatable occlusion balloon. U.S. Pat. No. 5,456,667 describes a temporary stent on a catheter. U.S. Pat. No. 5,733,294 describes a self-expanding cardiovascular occlusion device. U.S. Pat. Nos. 5,437,631, 5,591,204 and 5,383,897 describe a puncture wound sealer. U.S. Pat. No. 5,626,614 describes a tissue anchor for anchoring the stomach to the abdominal wall. U.S. Pat. No. 4,372,293 describes an instrument for the surgical correction of ptotic breasts. U.S. Pat. Nos. 5,730,733 and 5,336,205 describe flow-assisted catheters.

### BRIEF DESCRIPTION OF THE DRAWINGS

FIG. 1 is a mechanical schematic showing a device made according to this invention fully deployed in a plastic graft used in hemodialysis. The FIG. 1 drawing shows the blocking element at the distal end of the catheter in its radially expanded state and the occlusion engaging element at the distal end of the support wire in its radially expanded state. It is important to note that the blocking element may take a variety of shapes as would be required for the particular application. The preferred shape is likely to be a funnel shape where the larger diameter is distal to the lesser diameter that is proximal on the element. This funnel shape allows the obstruction to be more easily accepted into the catheter due to the pull/push of the engaging element, aspiration or both.

FIG. 2 is a longitudinal view of the distal portion of the support wire with a braided occlusion engaging element in its radial compressed state. This is the state where the support wire and engaging element can be inserted through the occlusion that is to be removed.

FIG. 3 shows the FIG. 2 braided occlusion engaging element in its radially expanded state, which is the state shown in FIG. 1.

FIG. 4 shows the multi-wing malecot type blocking element at the distal end of the catheter in its radially expanded state, which is the state shown in FIG. 1. It should be noted that the scale of the FIG. 4 catheter is much reduced compared to the scale of the occlusion removal wire and braided element shown in FIGS. 2 and 3.

FIG. 5 is a longitudinal view, in partial cross-section, showing the catheter and dilator with a ferrule at the distal tip of the guide wire in a passageway having an occlusion that is to be removed.

FIG. 6 shows the next step in which the dilator is being removed thereby causing the malecot type blocking mechanism to become expanded by virtue of pressure against the distal end of the catheter tip of the dilator.

FIG. 7 shows the next step in which the support wire together with the braided occlusion removal element in its radially compressed state (the state shown in FIG. 2) is inserted through the catheter and through the occlusion to be removed.

FIG. 8 shows the next step in which the braided occlusion removal element has been expanded and is being pulled in a proximal direction thereby forcing the occlusion into the catheter for removal with or without aspiration.

FIG. 9 shows the multi-wing malecot type blocking element at the distal end of the catheter in its radially expanded state in accordance with another embodiment of the present invention.

FIG. 10 shows the shape of the expansion resulting from the malecot type blocking element shown in FIG. 9.

FIGS. 11A-11C illustrate the use of a tissue removal assembly made according to the invention.

FIG. 12 shows the use of a sleeve which helps prevent seeding of a tissue track and provides access to a void within the patient.

FIGS. 13A-13H illustrate a further aspect of the invention by which percutaneous removal of target tissue from a target site within the patient is accomplished using a radially expandable/collapsible tubular shaft.

FIGS. 14A-14D show a method for percutaneously removing an entire tissue mass from a target site.

FIGS. 15A-15D illustrate a target tissue removing device including a pair of tissue engaging devices which bracket the target tissue.

FIGS. 16A-16C show the use of a pair of locational elements, one of which is left in place after target tissue is removed to provide guidance for re-access to the target site.

FIG. 17A illustrates a cross-sectional view of a patient's breast following removal of tissue at a target site, and illustrating a cavity created by the removed tissue, a sheath extending to the cavity, and an expandable element insertion device passing through the sheath into the cavity.

FIG. 17B illustrates an expanded expandable element within the void of FIG. 17A.

FIG. 17C-17E illustrate a loop type cutter, shown in more detail in FIGS. 18A-18F, separating a layer of tissue surrounding the expanded element.

FIG. 17F illustrates the removal of the separated layer of tissue with the aid of suction.

FIG. 17G illustrates an alternative to the use of suction in FIG. 17F using a radially expandable and contractible mesh material.

FIG. 17H illustrates the resulting cavity.

FIG. 17I illustrates an enlarged, simplified cross-sectional view of the layer of tissue removed during the steps of the FIGS. 17A-17H.

FIGS. 17J and 17K illustrate alternatives to the balloon-type expandable element of FIG. 17B.

FIGS. 18A-18F illustrate the opening and closing movements of the loop type cutter shown in FIGS. 17C-17E;

FIGS. 19A-19D illustrate the use of a radially expandable mesh type cutter to separate a layer of tissue surrounding a void having an expanded expandable element therein.

FIGS. 20A-C show the insertion of a flexible implant through a sheath providing access to a void within a patient's breast.

FIG. 21A illustrates placement of the suction inlet of a section device within a void at a target site within a patient.

FIG. 21B shows a blocking element shaft passing through the collapsed tissue at the target site, created by withdrawal of fluid through the suction device of FIG. 21A, and a radially expanded blocking element positioned distally of the target site.

FIGS. 21C-21E illustrate the positioning of a wire tissue cutter at the collapsed tissue of FIG. 21B, the radial expansion of the wire tissue cutter and the rotation of the wire tissue cutter to separate a layer of tissue surrounding the target site.

FIGS. 21F-21H illustrate passing a radially expandable, tubular mesh material between the separated layer of tissue and the surrounding tissue and then removal of the separated layer of tissue simultaneously with the removal of the tubular mesh material and the blocking element.

FIGS. 22A-22C illustrates an alternative to the method illustrated in FIGS. 21A-21H in which after the tissue has been collapsed using the suction device, as shown in FIG. 22B, a cutter element, such as illustrated in one or more of the above embodiments, is used to separate a layer of tissue surrounding the suction inlet of the suction device for removal from the patient.

FIG. 23 is a schematic illustration of a guide wire or catheter constructed in accordance with the principles of the present idea. FIG. 23-A is an illustration of the expandable guide wire or catheter in its relaxed un-deployed state (normally closed). FIG. 23-B is a schematic illustration of the expandable guide wire or catheter in its expanded state. FIG. 23-C is a schematic illustration of the ‘detached’ occluder.

FIG. 24 is a schematic illustration of the annular or tubular braid used in the instant invention.

FIG. 25 is a schematic illustration of the expanded braided ‘umbrella’ mechanism in place in a tubular channel of the body where the expanding element is used as an occluder, anchor, flow director or tensioner.

FIG. 26 illustrates the instant invention as it is being used as a detachable occluder. FIG. 26-A is a schematic illustration of the detached occluder in place in a tubular channel within the body. FIG. 26-B is a schematic illustration of the occluder being advanced in a tubular channel toward an aneurysm. FIG. 26-C is a schematic illustration of the detached occluder in place in the aneurysm. Although FIGS. 25 & 26 indicate use of the instant invention in a tubular channel of the body, it is recognized and disclosed heretofore that the instant invention has applicability toward many other areas other than those in the figures including, but not limited to anchoring the intestines or stomach, anchoring hearing aids, occlusion of any hollow structure, anchoring the bladder, anchoring the breasts to create a lifting force, anchoring the facial tissues to lift those tissues, etc. Further, although the instant invention in FIG. 23-B illustrates a relative motion of the inner and outer elongate member, it is recognized and disclosed heretofore that the expanding mechanism may be deployed any number of ways including, but not limited to self expansion (permanent set in the expanding mechanism that is constrained by an outer tubular channel prior to deployment, magnetic means, thermal gradient mechanisms, electrical stimulation, etc.

### DESCRIPTION OF THE PREFERRED EMBODIMENTS

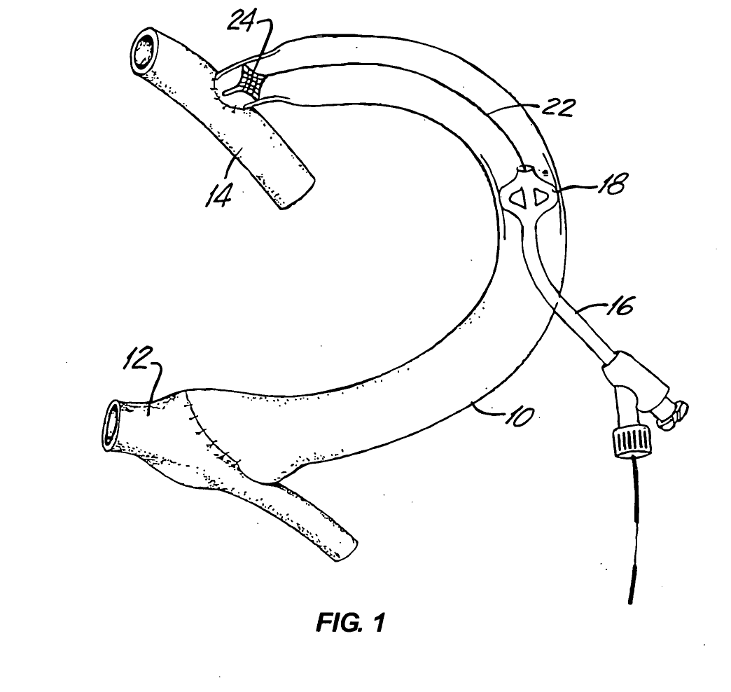

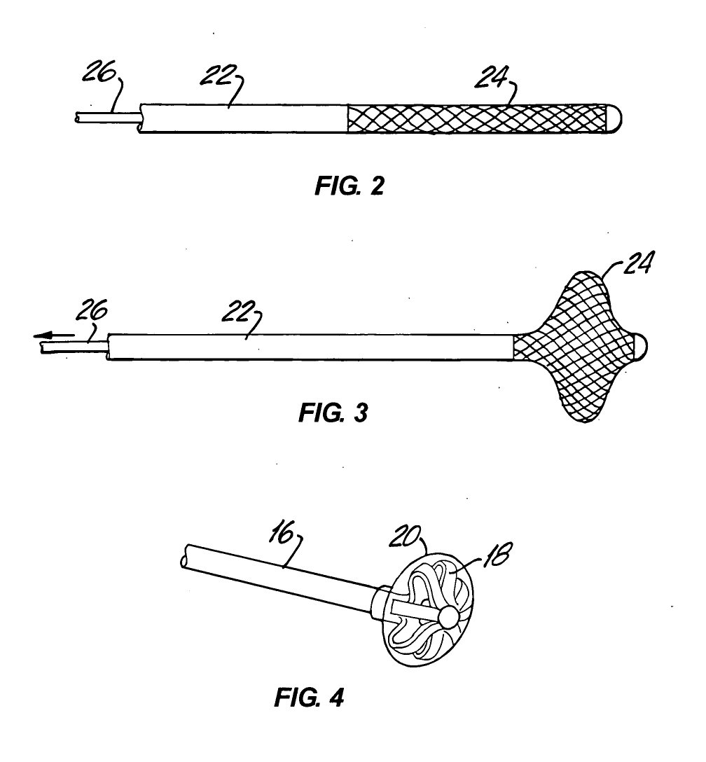

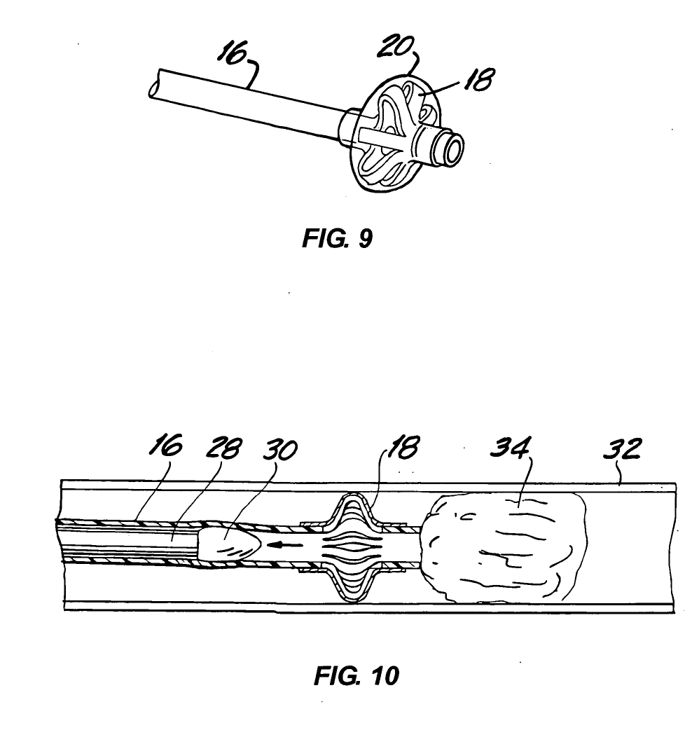

FIG. 1 shows a typical synthetic graft 10 used in hemodialysis. The graft extends between a vein 12 and an artery 14. The graft 10 may be about thirty centimeters long with an inner diameter (I.D.) of 6 or 7 millimeters. A catheter 16 is inserted through the wall of the graft or vessel. Typically the catheter might have an outside diameter (O.D.) of 2.7 mm and an inner diameter (I.D.) of 2.3 mm A malecot type expansion device 18 is covered with a membrane 20 (see FIG. 4). When expanded, it serves to block the annular space between the outside wall of the catheter 16 and the graft 10. A support wire 22 for a braided removal mechanism 24 will typically have an outside diameter of about one mm and has an internal actuator rod 26 (see FIG. 2) of approximately 0.5 mm. Because of the simplicity of the design, this outside diameter could be smaller than 0.5 mm. In FIG. 1, the malecot type blocking device 18 and the braided removal device 24 are both shown in their expanded state and are positioned so that retrograde or proximal movement of the support wire 22 will pull the braided element in a proximal direction to push out whatever coagulated blood is between the braided device 18 and the distal end of the catheter into the catheter opening where it can be aspirated; thereby clearing the blockage in the graft or other vessel.

More particular one embodiment of this invention which has been partly tested, was designed for use in a hemodialysis graft 10 having an I.D. of approximately six to seven mm. In that case, the catheter 16 has a 8 French O.D. (2.7 mm) and a 7 French I.D. (2.3 mm). The support wire 22 is a fairly standard movable core guide wire of 35 mils (that is, 0.35 inches, which is slightly under 1 mm). The actuator rod 26 in the support wire is approximately 15 mils and thus slightly under 0.5 mm. The braided element 24 has an insertion diameter that is approximately one mm and expands to cover the seven mm diameter of the graft. In order to achieve this seven fold increase in diameter, the braided element has a length of 11 to 13 mm Thus the catheter has an annulus of about 2.3 mm around the support wire, through which annulus the blood occlusion is aspirated.

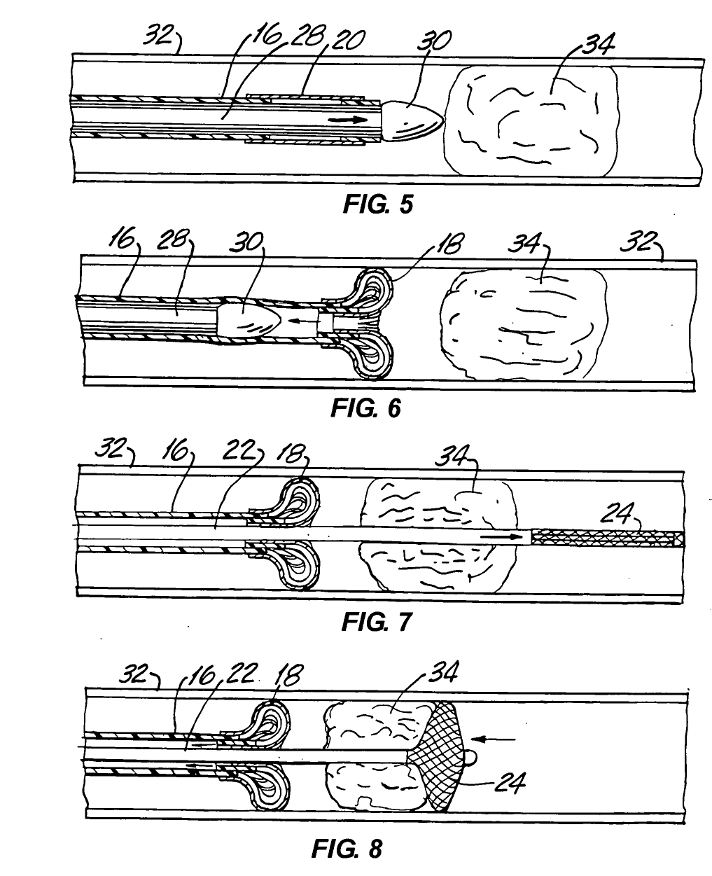

FIGS. 2 and 3 illustrate the support wire 22 and braided element 24 which constitute the occlusion engaging element that is moved proximal to push the occlusion into the catheter for removal. A preferred occlusion engaging element 24 is a braided element. The braided material has to have a stiffness such that it will not collapse or fold under the pressure of the occlusion when this engaging element is being moved proximally. Yet the filaments that form the braid must be flexible enough to be moved between the two states as shown in FIGS. 2 and 3. Materials from polyester to stainless steel can be successfully used. A more detailed teaching of the considerations that go into the selection of the braided engaging element is set forth fiber on.

The distal tip of the braided element 24 is connected to the distal tip of the actuator rod 26. The proximal edge of the braided element 24 is bonded to the distal end of the support wire 22. Thus when the actuator rod 26 is pushed in a distal direction relative to the wire 22, the braided device is forced into its collapsed state shown in FIG. 2 and is available to be pushed through the catheter and through or around the occlusion which is to be removed. When this engaging element 24 has been fully inserted, the actuator rod 26 is moved in a proximal direction causing the braided element 24 to take the expanded position such as that shown in FIG. 3 so that subsequent movement of the entire support wire 22 will cause the braided element to move against the occlusion and push the occlusion into the distal end of the catheter. In some circumstances, the braided element 24 might be left as a braid with openings because the portions of the occlusion which may pass through the openings will be sufficiently smaller liquids so that they do not have to be removed. In other circumstances, it might be desirable to cover the braided element 24 with a membrane or film so that it becomes substantially impermeable. Further the membrane or film covering the engaging element will be helpful in preventing trauma to the inner walls of native tissue. Even further, this membrane may be helpful in opting the physical characteristics of the engaging element.

With reference to FIG. 1, it might be noted that when the braided element is pushed all the way down to one end of the graft 10, as shown in FIG. 1, and then expanded it will be expanding against a portion of the wall of the graft that is smaller than the bulk of the graft. However, as the support wire 22 is pulled to move the braided occlusion removal element proximally, the braided occlusion element rides on the wall of the graft and will expand as the wall of the graft expands as long as tension is maintained on the actuator rod 26.

There might be applications of the invention where the passageway involved is a tissue passageway such as a blood vessel or other channel within the body, where this braided element 24 is expanded to nearly the diameter of the vessel so that when it is moved to push out an occlusion, it will avoid trauma to the wall of the vessel. Further, the membrane on the expanding element will aid in decreasing the trauma to native vessels as described above. In such a case, the engaging element (and the blocking element) may be used only as a ‘seal’ so that the obstruction may be removed or otherwise obliterated. This seal allows the rest of the vessel to be uncontaminated and provides for a ‘closed system’ for irrigation and/or aspiration and subsequent obliteration or removal of the obstruction

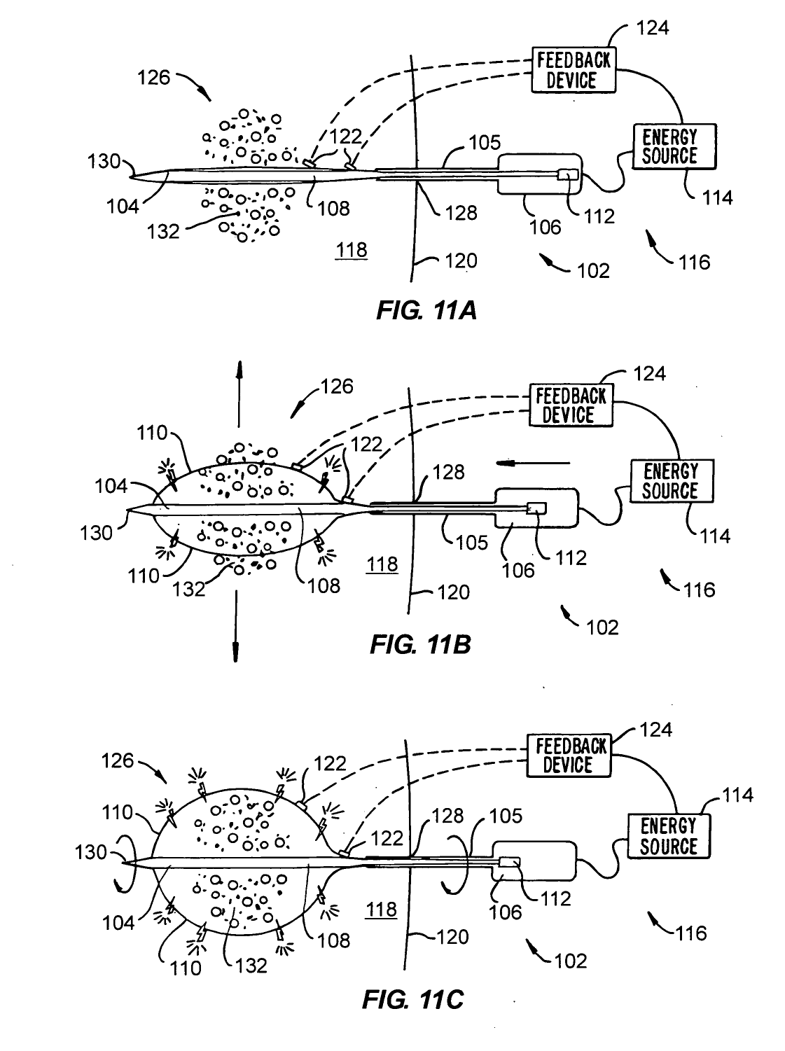

FIG. 4 illustrates the catheter 16 with the malecot 18 in an expanded state on the distal end of the catheter. A membrane 20 is normally used in order to provide a complete blocking or sealing function. Further, the membrane 20 may aid in locking the blocking element in a particular shape. This malecot type element is created by making longitudinal slits in the sidewall of the catheter (or an attachment bonded thereto) thereby creating links or wings that will expand when the distal end of the catheter is pushed in a proximal direction. The appropriate pushing of the proximal end of the catheter is achieved, as shown in FIG. 5, by a ferrule 30 which is a standard tip on a standard dilator 28. Alternatively, the dilator 28 may be a guide wire (which is usually much longer and flexible than a dilator) for remote obstruction removal. In such an application of the present invention, the guide wire would have a ferrule type mechanism that would act like the ferrule on the dilator. In this instance, the guide wire (with ferrule) would be inserted into the vessel to the obstruction. The catheter would then be pushed along the guide wire until it reached the ferrule which would normally be located near the distal end of the guide wire. At this point the wire would be pulled back, the ferrule would butt against the catheter and force out the blocking sealing element. The engaging element may be used with this blocking element and it could even be the ferruled wire as well.

It should be noted that the retention catheter described in U.S. Pat. No. 3,799,172 issued on Mar. 26, 1974 to Roman Szpur illustrates a structure that is similar to the malecot type device 18 illustrated in FIG. 4; although in that patent it is used as a retention device whereas in this invention it is used as a blocking element.

This blocking element 18 is often called a malecot in the industry. It should be understood herein that the term malecot is used to refer in general to this type of multi-wing device.

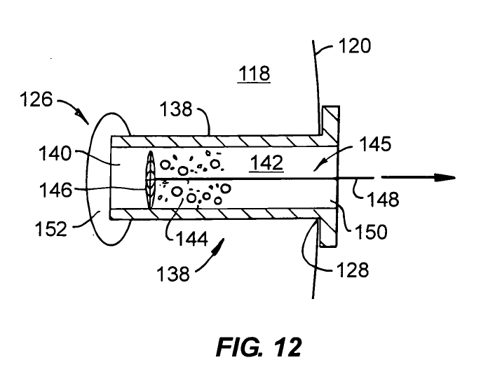

More specifically, as shown in FIG. 5, the catheter 16 together with a dilator 28 having an expanded tip 30 which is a ferrule is inserted into a vessel 32 such as the graft shown in FIG. 1. The catheter 16 and dilator 28 are inserted close to the occlusion 34 and then the dilator 28 is removed. Proximal motion of the dilator 28 causes the tip 30 to contact the distal end of the catheter 16 forcing the distal end of the catheter to put pressure on the malecot wings creating the expansion shown in FIG. 6 (and also schematically shown in FIG. 1). Once this expansion has occurred, the dilator with its tip can be removed from the catheter (as shown in FIG. 6).

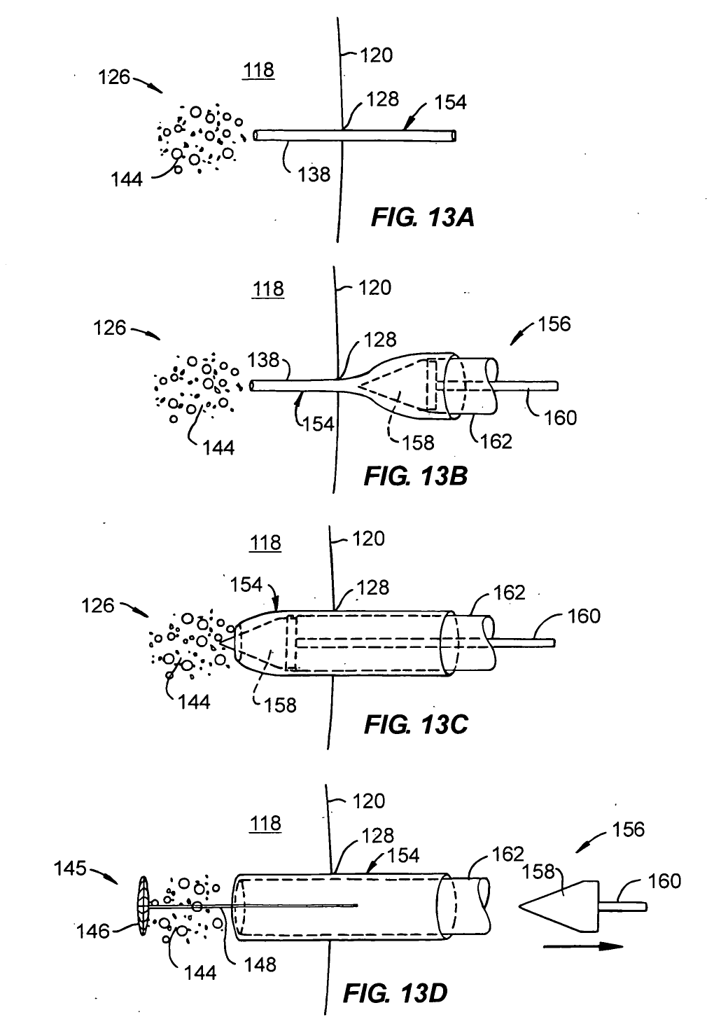

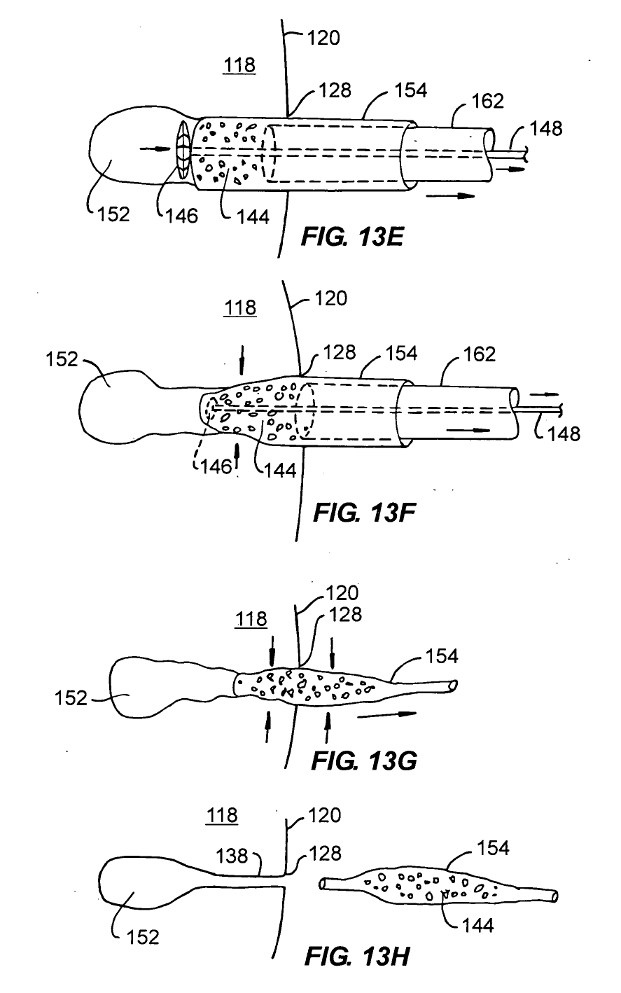

What then occurs is shown in FIGS. 7 and 8. As shown in FIG. 7, the support wire 22 with its braided removal element 24 is inserted in the collapsed state so that it passes through or around the occlusion 34. It should be noted that the support wire 24 may be inserted prior to the blocking catheter being inserted or after the catheter is inserted (the latter of which is illustrated in the figures). Most of the occlusions to which this invention is directed such as congealed blood in a graft will permit a support wire 22 to pass through it because the consistency is that of viscous material which can be readily penetrated. Alternatively, if the occlusion is a non viscous material such as a stone, plaque, emboli, foreign body, etc. the support wire 22 is small enough to be passed around the occlusion. Once the braided element 24 is on the distal side of the occlusion 34, the actuator rod 26 is pulled creating the expanded state for the braided device. Accordingly, distal movement of the entire support wire will cause the expanded braided device to move against the occlusion and force it into the catheter for removal with or without aspiration. When removal of obstructions that are located some distance array from the point of access into the body such as the carotid artery via a groin access the wire 22 would likely be inserted first. In this case the support are 22 with its expanding element 24 may be used as a guide wire to guide the catheter to the preferred location. Of further import is that the blocking element and the engaging element may be used without any relative motion once deployed. Such is the case when irrigation and/or aspiration is used for the obstruction removal In this case the two elements can be used as seals against the tubular inner walls on both sides of the obstruction whereby the obstruction is removed from that ‘sealed’ space with the use of aspiration, irrigation, or both. Further other means of obliterating the obstruction within this ‘sealed’ space may be employed. Some of those means are, but are not limited to the addition of dissolving agents, delivery of energy such as ultrasound, laser or light energy, hydraulic energy and the like.

The Tubular Braid Engaging Element

The engaging apparatus includes an elongate tube; an elongate mandril inside the tube and an expandable tubular braid. The elongate mandril extends from the proximal end of the device to the distal end. The elongate tube extends from close to the proximal end of the device to close to the distal end. The distal end of the tubular braid is bonded to the distal end of the inner elongate mandril. The mandril may extend beyond the tubular braid. The proximal end of the tubular braid is bonded to the distal end of the elongate tube.

The braid may be open, but may be laminated or covered with a coating of elastic, generally inelastic, plastic or plastically deformable material, such as silicone rubber, latex, polyethylene, thermoplastic elastomers (such as C-Flex, commercially available from Consolidated Polymer Technology), polyurethane and the like. The assembly of tube, mandril and braid is introduced percutaneously in its radially compressed state. In this state, the outside diameter of the braid is close to the outside diameter of the elongate tube. This diameter is in the range of 10 to 50 mils, and usually 25 to 40 mils (i.e. thousandth of an inch). After insertion, the tubular braid is expanded by moving the mandril proximally with respect to the tube.

The tubular braid is preferably formed as a mesh of individual non-elastic filaments (called “yarns” in the braiding industry). But it can have some elastic filaments interwoven to create certain characteristics. The non-elastic yarns can be materials such as polyester, PET, polypropylene, polyamide fiber (Kevlar, DuPont), composite filament wound polymer, extruded polymer tubing (such as Nylon II or Ultem, commercially available from General Electric), stainless steel, Nickel Titanium (Nitinol), or the like so that axial shortening causes radial expansion of the braid. These materials have sufficient strength so that the engaging element will retain its expanded condition in the lumen of the body while removing the obstruction therefrom.

The braid may be of conventional construction, comprising round filaments, flat or ribbon filaments, square filaments, or the like. Non-round filaments may be advantageous to decrease the axial force required for expansion to create a preferred surface area configuration or to decrease the wall thickness of the tubular braid. The filament width or diameter will typically be from about 0.5 to 25 mils, usually being from about 5 to 10 mils. Suitable braids are commercially available from a variety of commercial suppliers.

The tubular braids are typically formed by a “Maypole” dance of yarn carriers. The braid consists of two systems of yarns alternately passing over and under each other causing a zigzag pattern on the surface. One system of yarns moves helically clockwise with respect to the fabric axis while the other moves helically counter-clockwise. The resulting fabric is a tubular braid. Common applications of tubular braids are lacings, electrical cable covers (i.e. insulation and shielding), “Chinese hand-cuffs” and reinforcements for composites. To form a balanced, torque-free fabric (tubular braid), the structure must contain the same number of yarns in each helical direction. The tubular braid may also be pressed flat so as to form a double thickness fabric strip. The braid weave used in the tubular braid of the present invention will preferably be of the construction known as “two dimensional, tubular, diamond braid” that has a 1/1 intersection pattern of the yarns which is referred to as the “intersection repeat”. Alternatively, a Regular braid with a 2/2 intersection repeat and a Hercules braid with an intersection repeat of 3/3 may be used. In all instances, the helix angle (that being the angle between the axis of the tubular braid and the yarn) will increase as the braid is expanded. Even further, Longitudinal Lay-Ins can be added wit the braid yarns and parallel to the axis to aid with stability, improve tensile and compressive properties and modulus of the fabric. When these longitudinal “Lay-In” yarns are elastic in nature, the tubular braid is known as an elastic braid. When the longitudinal yarns are stiff, the fabric is called a rigid braid. Biaxially braided fabrics such as those of the present invention are not dimensionally stable. This is why the braid can be placed into an expanded state from a relaxed state (in the case of putting it into the compressive mode). Alternatively this could be a decreased/reduced (braid diameter decreases) state when put into tension from the relaxed state. When put into tension (or compression for that matter) the braid eventually reaches a state wherein the diameter will decrease no more. This is called the “Jammed State”. On a stress strain curve, this corresponds to increase modulus. Much of the engineering analysis concerning braids are calculated using the “Jammed state” of the structure/braid. These calculations help one skilled in the art to design a braid with particular desired characteristics. Further, material characteristics are tensile strength, stiffness and Young's modulus. In most instances, varying the material characteristics will vary the force with which the expanded condition of the tubular can exert radially. Even further, the friction between the individual yarns has an effect on the force required to compress and un-compress the tubular braid. For the present invention, function should be relatively low for a chosen yarn so that the user will have little trouble deploying the engaging element. This is particularly important when the engaging element is located a significant distance from the user. Such is the case when the percutaneous entry is the groin (Femoral Artery for vascular interventions) and the point of engaging the engaging element is some distance away (i.e. the Carotid Artery in the neck). Similarly, this is true for long distances that are not vascular or percutaneous applications.

Other Comments

An important consideration of the invention described herein is that the support wire with its expanding element can be fabricated with a very small diameter. This is important because it allows an optimally large annular space between the wire and the inside of the catheter for maximum obstruction removal. Previous engaging elements have been used that use a balloon for the engaging element. This balloon design requires a larger shaft diameter than that of the present invention. Hence in these previous devices the annular space is not maximized as in the present invention. The term wire is used to refer to the support portion of the removal device. The material of the wire need not necessarily be metal. Further, it may be desirable to use a ‘double’ engaging element (i.e. two braided or malecot expanding elements separated a distance appropriate to entrap the occlusion) in the case for example where the occlusion is desired to be trapped in the vessel. The term wire is used herein to refer to a dual element device having a shell component and a core or mandril component which are longitudinally moveable relative to one another so as to be able to place the braided occlusion engaging element into its small diameter insertion state and its large diameter occlusion removal state.

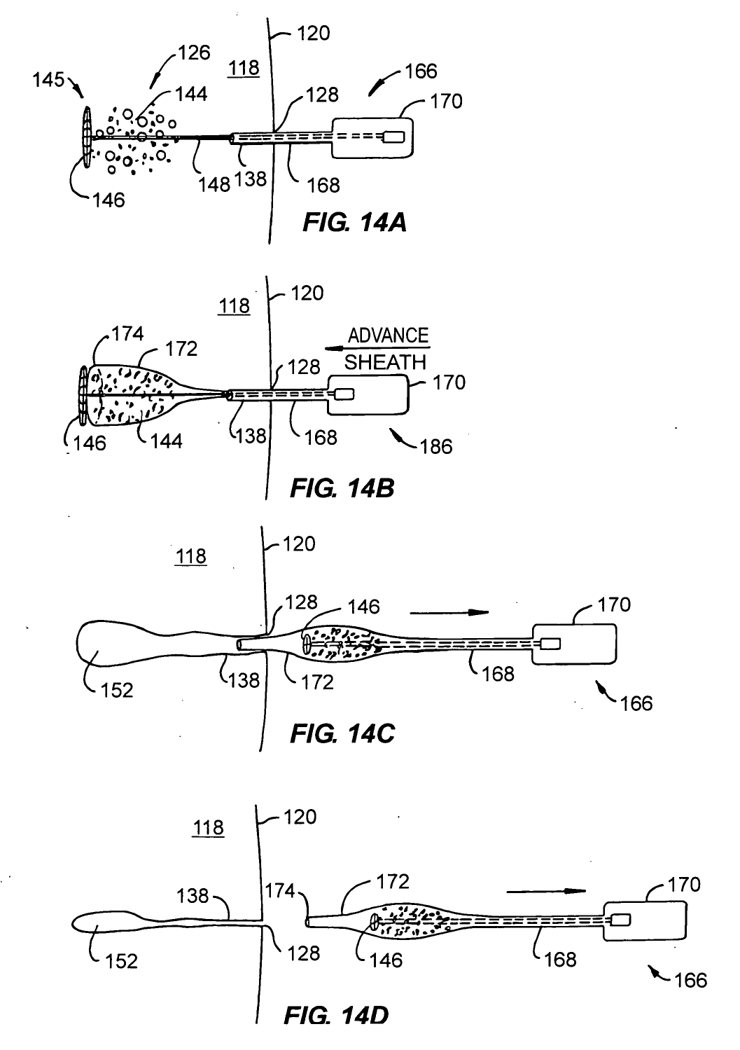

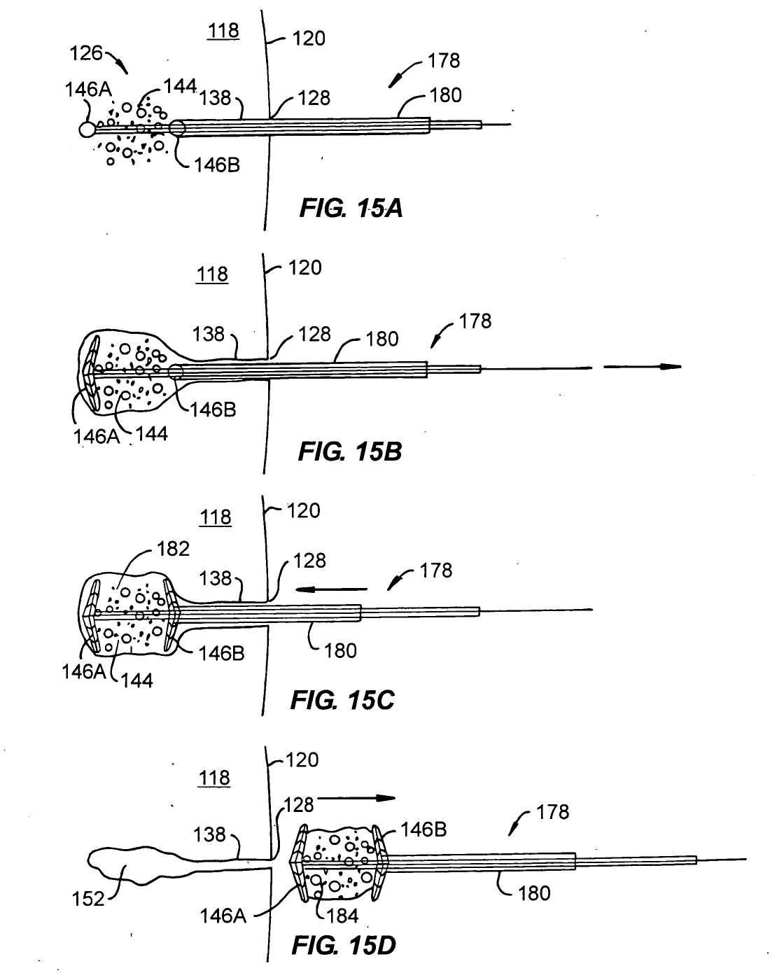

Although the blocking element is described as a multi-malecot type of device, it should be understood that the blocking element may be designed in various fashions which are known in the art. See, for example, FIGS. 9 and 10. As another example, an appropriately designed braid arrangement could be used as the blocking element. In that case, the catheter may have to be a dual wall catheter in which the inner and outer annular walls are able to move relative to one another in a longitudinal direction so as to place the braid used as a blocking element in its small diameter insertion state and its large diameter blocking state. Alternatively, it may be a single wall similar in design to the malecot style blocking element described previously.

The particular embodiment disclosed was designed for an application to remove congealed blood in a dialysis graft. For some applications, like removing clots from remote vascular areas, the blocking mechanism and engaging elements may be used only as distal and proximal seals around the device to be removed so that the clot or other obstruction can be removed with aspiration or can be obliterated with some therapy such as a chemical dissolving agent or acoustical energy or lithotripsy and the like. The residual obstruction in that case would be aspirated from the tubular catheter.

It should be further understood that there might be a situation in which the blocking element or even the occlusion engaging element would be provided to the physician in a normal expanded state so that when the device is deployed, it would, through plastic memory or elastic memory, automatically snap into its expanded state.

The Tissue Removal Assembly

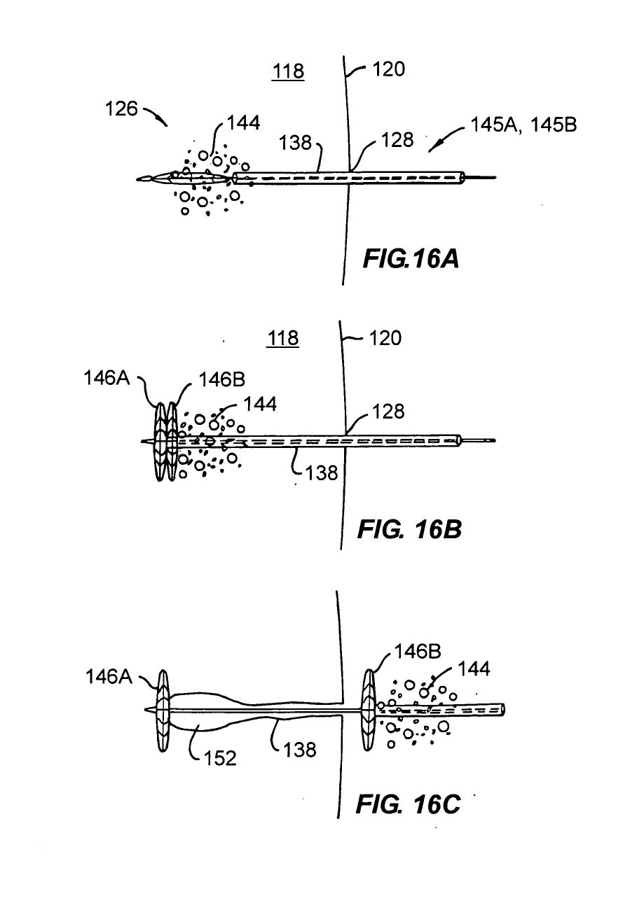

FIGS. 11A-11C illustrate the use of a tissue removal assembly 102. Tissue removal assembly 102 includes a support shaft 104 passing through an introducer sheath 105 extending from a handle 106. The distal portion 108 of shaft 104 has a pair of tissue separation wires 110 mounted thereto. Wires 110 are movable from a retracted state of FIG. 11A to a fully extended state of FIG. 11C by moving a slide 112 mounted to handle 106 as indicated in FIGS. 1A-1C. Wires 110 are typically made of tungsten or stainless steel and may have a round, rectangular or other cross-sectional shape depending upon the type of tissue and other matter expected to be encountered. U.S. Pat. No. 6,221,006 and Provisional Application 60/154,394 (filed Sep. 17, 1999 and entitled Oncological Apparatus and Method for Use) and 60/200,546 describe various tissue separation elements. Wires 110 are coupled to an energy source 114 to supply wires 110 with appropriate energy to aid the cutting or other separating actions of the wires, including electrical, RF, vibrational, electromagnetic, etc. Together, handle 106 and energy source 114 constitute a wire tissue separation element driver 116 because both act to help move wires 110 through tissue 118 beneath a skin surface 120 of the patient.

Appropriate sensors 122 are mounted to one or more of wires 110 and shaft 104. Sensors 122 could be portions of wires 110 themselves. Sensors 122 may include strain gauge sensors, pressure sensors, temperature sensors, etc. Sensors 122 are coupled to a feedback device 124 through sheath 105; feedback device 124 is connected to energy source 114 to ensure that energy source 114 provides an appropriate level of energy to wires 110.

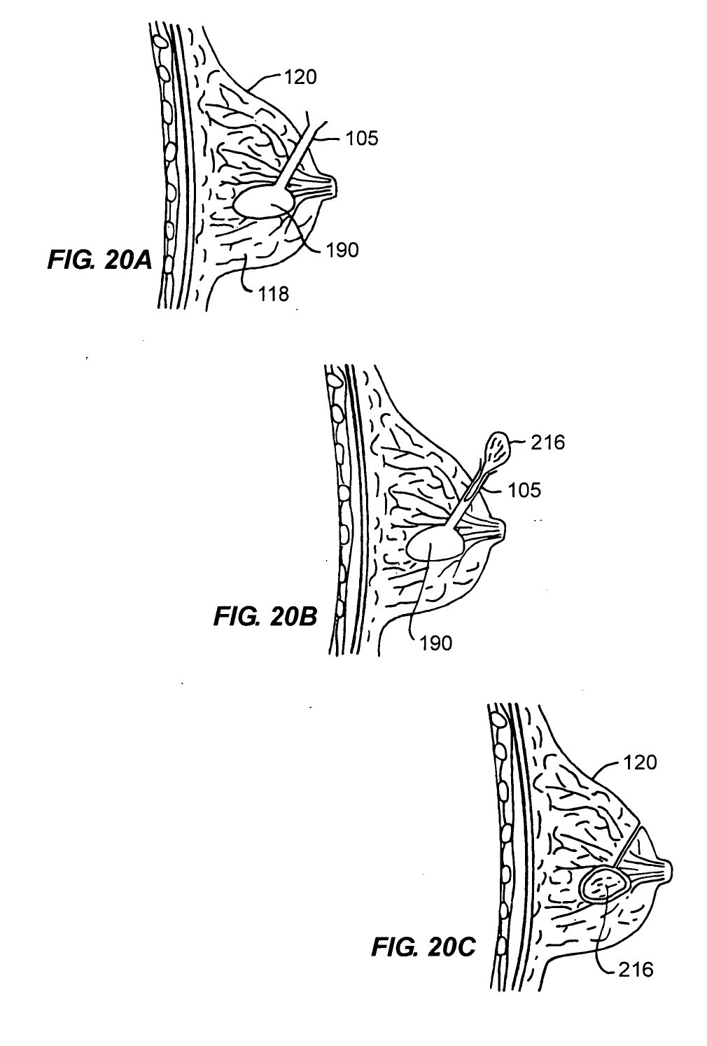

Assembly 102 is used to percutaneously access a target site 126 through an access site 128 in skin surface 120 while in the retracted state. The tip 130 of shaft 104 is positioned distally of the target tissue mass 132. In some situations it may be desirable to pass tip 130 directly through target tissue mass 132 while in other situations it may be desirable to have shaft 104 pass to one side of target tissue mass 132 or proximal to the tissue mass as in FIGS. 17A-19D. Once properly positioned, which is preferably accomplished with the aid of remote visualization techniques, such as x-rays, ultrasound, etc., slide 112 is moved in a distal direction causing wires 110 to arc outwardly from the retracted state of FIG. 11A, through the intermediate extended state of FIG. 11B and to the fully extended state of FIG. 11C. Wires 110 are preferably energized, typically by heating using resistance or RF heating techniques, as wires 110 pass through tissue 118. This is very important when wires 110 pass through target tissue mass 132 and the target tissue mass contains, or possibly contains, cancerous or other diseased tissue. By appropriately energizing wires 110, the tissue wires 110 pass through is, for example, cauterized so that no viable diseased tissue is pulled along with the radially outwardly expanding wires; this helps to keep the healthy tissue surrounding target tissue mass 132 free from viable diseased tissue. In addition to heating or vaporizing the tissue, tissue removal assembly 102 may be provided with vibrational, reciprocating or other mechanical energy to help passage of wires 110 through tissue 118.

Once fully expanded, tissue removal assembly 102 is rotated, typically by the user manually grasping and rotating handle 106. If desired, a motorized or other non-manual rotation of assembly 102 could be provided for. Sensors 122 provide appropriate information to feedback device 124 so to ensure a proper amount of energy is supplied to wires 110 to, among other things, ensure proper cauterization of the tissue as wires 110 are moved readily outwardly while not overly damaging the tissue. Therefore, if wires 110 cease to be driven and thus stop moving through the tissue, feedback can result in a halt in the supply of energy to wires 110. Once in the fully extended state of FIG. 11C, the amount of energy supplied to wires 110 may not need to be as great as when, for example, wires 110 pass through only healthy tissue.

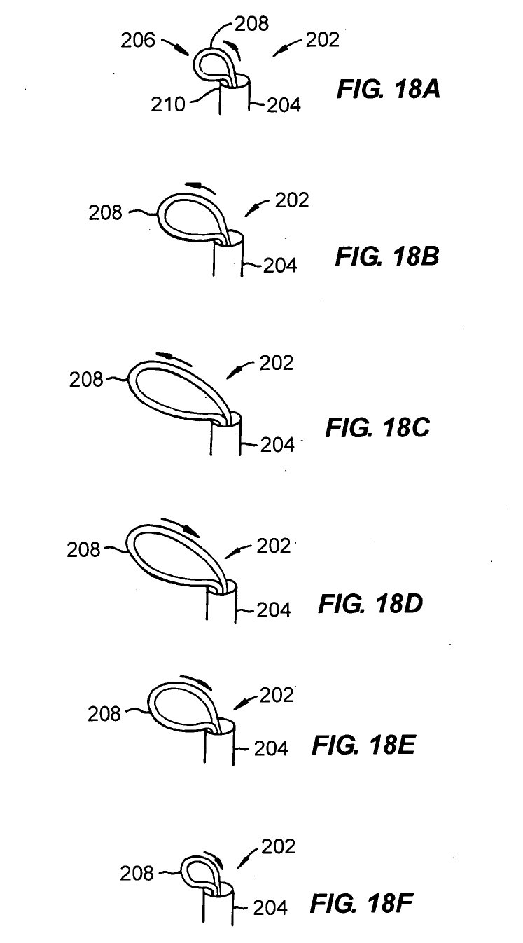

In the embodiment of FIGS. 1A-11C two wires 110 are used. This causes target tissue mass 132 to be cut away from the surrounding tissue in two contiguous tissue masses. If desired, only a single wire 110 or more than two wires 110 could be used. The number of wires may be limited to, for example, 3 or 4 so that the sections removed are large enough to be identifiable. However, if one were to put additional wires into the assembly, even if only one wire was used for severing the tissue, the additional wires may help with removal of the tissue as they may be used to encapsulate the tissue. Using the method described with respect to FIGS. 11A-11C, the entire target tissue mass 132 may be removed in a simultaneous manner. This aspect of the invention will be described in more detail below with reference to FIGS. 14A-14D. All or part of the procedure, such as expanding, cutting, rotating, energizing, etc., could be automated.

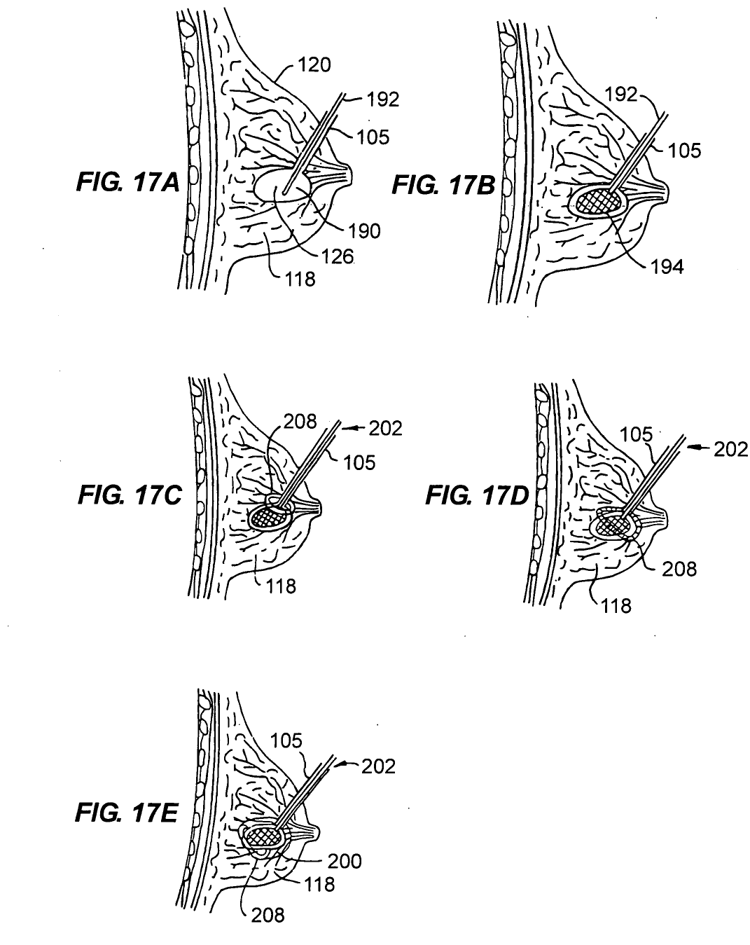

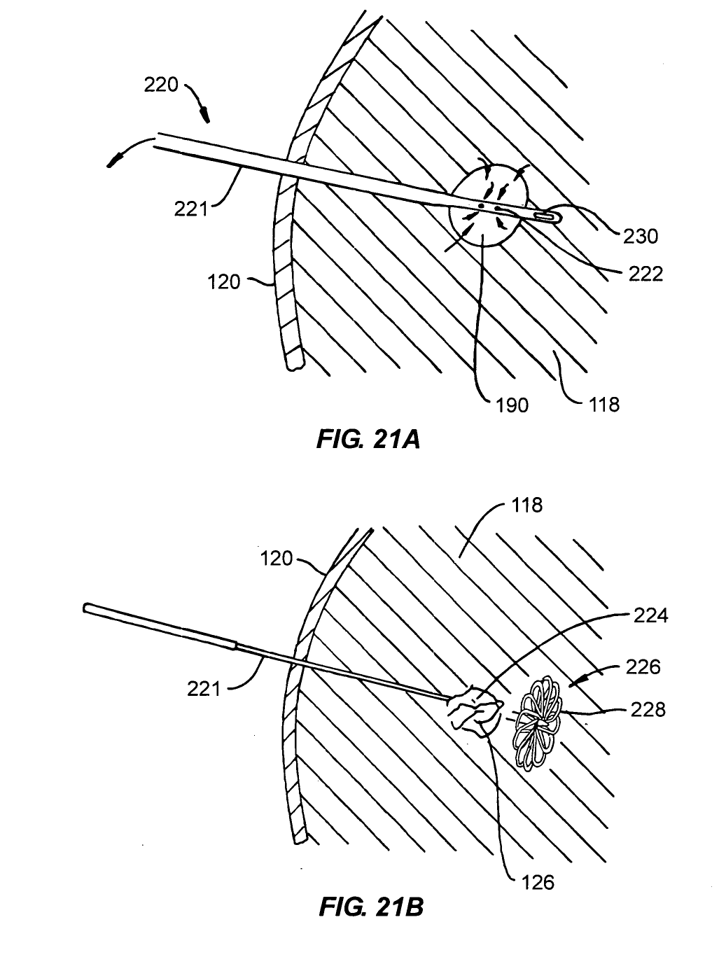

FIG. 12 illustrates a sleeve 136 used to help prevent seeding of a tissue track 138 extending between access site 128 and target site 126. Protective sleeve 136 is positioned along tissue track 138 and has a distal opening 140, preferably positioned adjacent to or within target site 126, and an open interior 142. Target tissue 144 is moved from target site 126 through opening 140 and into open interior 142. FIG. 12 illustrates this having been accomplished using a tissue engagement device 145 having a radially expandable mesh device 146 at the distal end of a shaft 148. Mesh device 146 is of a type which can be movable from a generally cylindrical orientation, not shown, to the radially extended configuration shown in FIG. 12 by pushing the distal ends of the cylindrical mesh material towards one another. Examples of this type of mesh structure can be found in U.S. Pat. No. 6,179,860 and in Provisional Application No. 60/200,546. Other methods and devices for moving target tissue 144 from target site 126 into interior 142 can also be used. Alternatively, the end of sleeve 136 could be used to sever the tissue while sleeve 136 is moved forward and a cutting/separating snare, see FIGS. 18A-18F, could separate the distal side of the tissue. Target tissue 144 can then be removed from the patient by either leaving protective sleeve 136 in place and sliding the target tissue out through the opened proximal end 150 of sleeve 136 or by removing the entire structure, that is protective sleeve 136, mesh device 146, shaft 148 and target tissue 144 therewith, from tissue track 138 of the patient. Suction may also be used to remove tissue. Removed tissue may be analyzed to see if additional tissue needs to be removed.

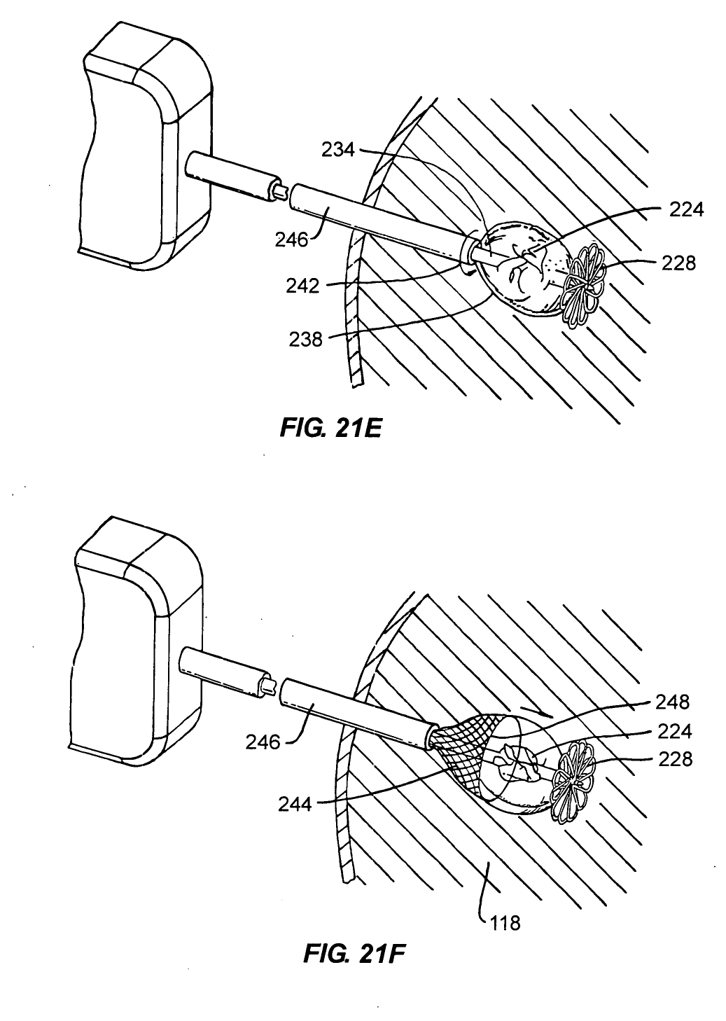

Access to a void 152 within a patient can be maintained by placing sleeve 136 along tissue track 138 and leaving it in place. This method may be accomplished after removal of, for example, a biopsy specimen or an entire suspect tissue mass. This provides convenient and accurate re-access to void 152. Such re-access may be used, for example, when additional tissue samples are needed, therapeutic agents (including heat treatment agents, mechanical treatment agents, chemical agents and radioactive agents) need to be delivered to void 152, a prosthesis is to be implanted into void 152, or for other reasons. See the discussion below with reference to FIGS. 20A-20C.

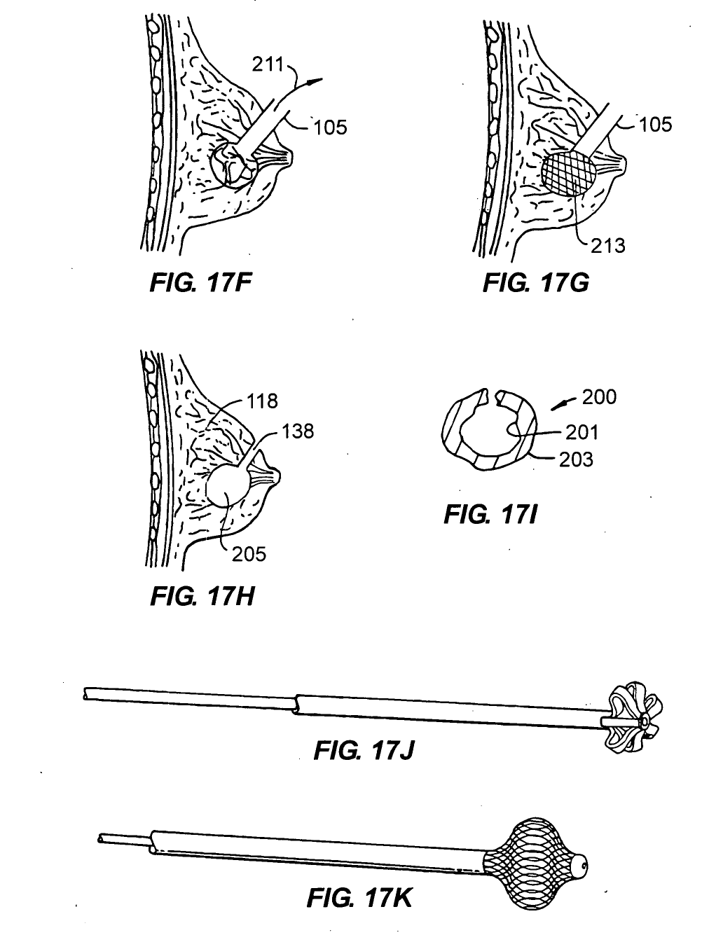

FIGS. 13A-13H illustrate the percutaneous removal of target tissue 144 from target site 126. A hollow, radially expandable/collapsible tubular shaft 154 is passed along tissue track 138 when in a radially collapsed condition as shown in FIG. 13A. FIG. 13B illustrates the introduction of a tubular enlarger 156 including a conical tip 158 mounted to the distal end of a shaft 160 and a stabilizing sleeve 162 extending proximally from conical tip 158. As illustrated in FIGS. 13B and 13C, pushing enlarger 156 through shaft 154 causes the shaft to radially enlarge along its length; stabilizing sleeve 162 resists the tendency of shaft 154 to radially collapse. Once sleeve 162 is properly positioned within shaft 154, shaft 160 and tip 158 therewith are removed from within sleeve 162 as shown in FIG. 13D. Also, FIG. 13D illustrates the positioning of a tissue engagement device 145 to help draw a sample of target tissue 144 into the interior 164 of sleeve 162 as suggested in FIGS. 13D and 13E.

At this point a sample of the target tissue 144 may be removed from the patient by simultaneously removing shaft 154 in its enlarge diameter form, sleeve 162 and device 145 as a unit. Alternatively, stabilizing sleeve 162 may be removed as device 145 pulls tissue 144 into shaft 154 while shaft 154 remains in place. This suggested in FIGS. 13E and 13F and permits shaft 154 to return towards its initial, radially contracted condition thus causing the tissue sample housed therein to be radially compressed. The collected target tissue 144 remains within shaft 154 when sleeve 162 is removed from shaft 154 and mesh device 146 is collapsed (see FIG. 13F). Shaft 154 then naturally assumes a smaller diameter condition as shown in FIGS. 13F and 13G which permits shaft 154 and the target tissue therein to be removed through access site 128 as shown in FIGS. 13G and 13H. In this way the size of access site 128 may be smaller than the original size of target tissue 144. Device 145 may remain within shaft 154 during this removal from the patient, or device 145 may, as suggested in FIGS. 13G and 13H, be removed from shaft 154 along with sleeve 162. Alternatively, mesh device 146 may not be required as mentioned above.

The entire shaft 154 was enlarged in the embodiment of FIGS. 13A-13H. If desired, only the part of shaft 154 within the patient may need to be expanded. This would reduce the maximum size which access site 128 is forced to assume, even if only temporarily. The following U.S. patents show radially-expanding dilators: U.S. Pat. Nos. 5,183,464; 5,431,676; 5,454,790.

FIGS. 14A-14D illustrate a method for percutaneously removing an entire tissue mass containing target tissue 144. A tissue removal assembly 166 includes a sheath 168 extending from a proximal end adapter 170 and passes through an access site 128 and along tissue track 138. Sheath 168 houses a tissue engagement device 145, shown in FIG. 14A, after having passed by or through target tissue 144 and manipulated to cause mesh device. 146 to assume a radially expanded condition. Next, a tubular mesh device 172, or other suitable mechanism, is used to surround target tissue 144. Device 172 is of the type in which a tubular mesh material having an open distal end expands radially outwardly as it is compressed axially. That is, the resistance to the axial movement mesh device 172 causes it to contract axially and expand radially to assume the generally funnel-shaped configuration of FIG. 14B. As shown in FIG. 14B, mesh device 146 acts as a blocking element and mesh device 172 acts as a removing element. Together devices 146, 172 at least substantially surround, and preferably fully surround or envelope, target tissue 144.

The entire suspect tissue mass, that is the mass including target tissue 144 and an amount of surrounding tissue (or only a portion of target tissue 144, such as for biopsy), can be removed through access site 128. To help prevent trauma to access site 128 during such removal, mesh device 146 and tubular mesh device 172 are caused to contract radially, thus compressing target tissue 144 into a smaller diameter mass for ease of removal from the patient. This is suggested in FIGS. 14C and 14D. The construction and use of structure similar to device 172 is described in U.S. Pat. No. 6,221,006 and Provisional Application No. 60/200,546. Note that the structure shown in FIGS. 11A-11C could be used to severe target tissue 144 so that the entire suspect tissue mass (or a part of the suspect tissue mass, such as for biopsy), that is including target tissue 144, may be simultaneously removed as two contiguous pieces from the patient along the tissue track. It is expected that the entire suspect tissue mass could be severed into at most four contiguous pieces and still be simultaneously removed in a useful condition for further testing and/or evaluation. One such structure could use the cutting device of FIGS. 11A-11C plus a mesh material similar to tubular mesh device 172 which could be guided by expanded wires 110 to surround the suspect tissue mass. As seen by comparing FIGS. 14B and 14C, the largest lateral dimension of the access opening 128 is smaller than the largest lateral dimension of a suspect tissue mass prior to removal; radially or laterally squeezing the suspect tissue mass permits removal of the tissue mass with minimal trauma to the patient. The suspect tissue mass may be monitored for disease prior to, during and/or after removal from the patient.

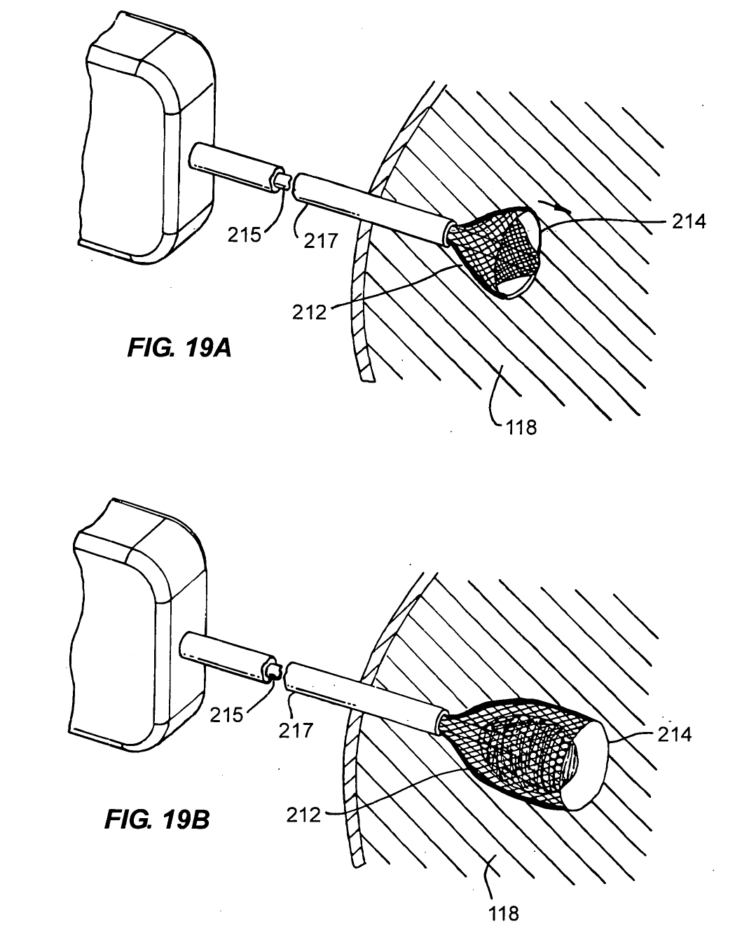

FIGS. 15A-15D illustrate a target material removing device 178 including a sheath 180 within which a pair of tissue engaging devices 145 slidable pass. FIG. 15A illustrates device 178 passing through access site 128, along tissue track 138 and to target tissue 144 at target site 126. The first and second mesh devices 146A, 146B are placed at distal and proximal locations relative to target tissue 144. Once in position, mesh devices 146 are expanded as shown in FIGS. 15B and 15C so to bracket target tissue 144. Mesh devices 146A, 146B in their expanded conditions are sized so to define a bracketed region 182 therebetween. Bracketed region 182 is preferably sized to completely contain the tissue mass including target tissue 144. When so bracketed, the health professional can locate target tissue 144 by virtue of the expanded mesh devices 146. In one embodiment mesh devices 146A, 146B are harder than the surrounding tissue so that target tissue 144 within bracketed region 182 may be found by palpation. In addition, expanded meshed devices 146A, 146B guide a surgeon in locating and excising the entire target mass using surgical techniques. The using of bracketing guides 146A, 146B is important because target tissue 144 is often difficult to differentiate from surrounding tissue both in appearance and in feel. After the surgeon has accessed target tissue 144, guided by bracketing mesh devices 146, the entire suspect tissue mass 184 can be removed as a single mass as suggested in FIG. 15D. It is expected that the device of FIGS. 15A-15D may be useful in both percutaneous and open incisional situations. Note that bracketing mesh devices 146A and 146B may be designed so that they are shaped like cones or funnels so that their opposed edges meet to sever and capture suspect tissue mass 184 therebetween.

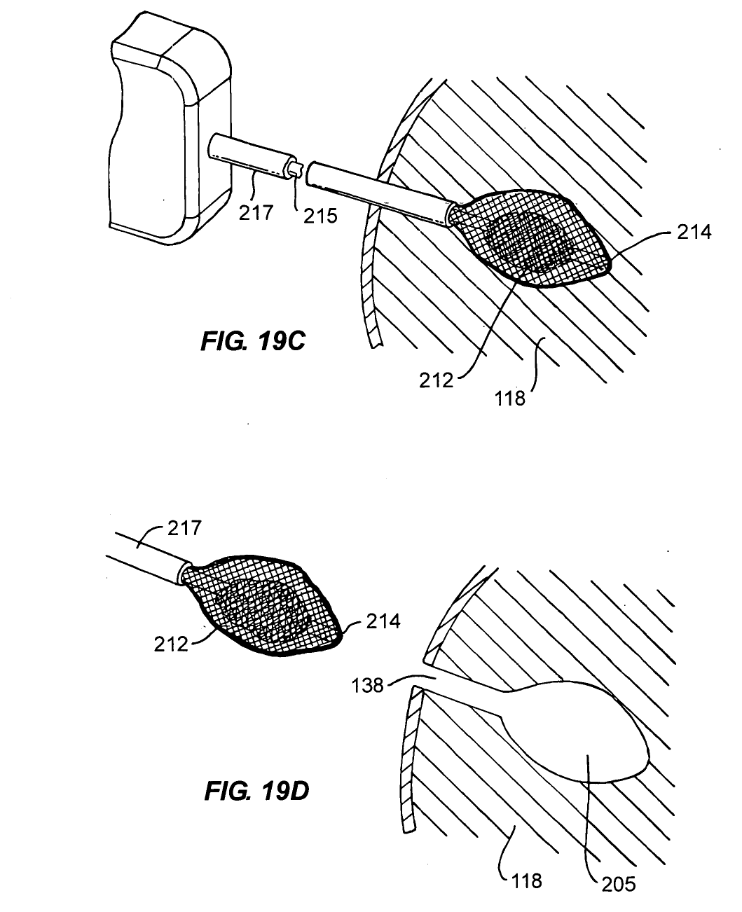

FIG. 16A-16C show the use of essentially the same type of structure as in FIGS. 15A-15D but for a different purpose. In this case devices 145 are used as locational elements. In the preferred embodiment both of the locational elements have radially expandable elements, such as mesh devices 146, both of which are positioned distally of target tissue 144. After removal of target tissue 144, which may occur along with proximal device 145B, device 145A remains in place adjacent to the excisional site or void 152 created by the removal of target tissue 144. This may be used to help maintain void 152 open to aid re-access to the site. Maintaining void 152 open also permits insertion of a space-saving device or structure into void 152. Instead of using two radially expandable elements as portions of the locational devices, locational device 145A could be simply, for example, a catheter shaft in which with the distal end would remain at the distal end of excisional site 152.

Turning now to FIGS. 17A-22C, with like reference numerals referring to like elements, further aspects of the invention, relating to intraoperative tissue treatment methods, will be discussed. The treatment methods are designed to be intraoperative, that is practiced closely following the removal of target tissue from a target site, typically within a patient's breast, leaving access to the target site, such as introducer sheath 105 being left along tissue track 138.

FIG. 17A illustrates a void 190 at target site 126 being accessed by an expandable element insertion device 192 through sheath 105. FIG. 17B shows an expanded balloon 194 at the distal end of insertion device 192 in an expanded condition substantially filling void 190. Balloon 194, or some other expandable element such as an expandable malecot 196 (FIG. 17J) or an expandable braided element 198 (FIG. 17K) may be expanded to a size greater that of void 190 thus expanding the void slightly. It may be desired to do this to compress the surrounding tissue to facilitate subsequent removal of a layer of tissue 200 from surrounding the expandable element 194 or for other reasons. The tissue that creates void 190 is tested to determine if all the target tissue, typically diseased tissue, has been removed. If it is determined that all of the target tissue has been removed, then the patient is closed in the usual fashion. However, there may be a need for access for additional or adjunctive therapy. Even further, another material or an implant may be placed inside the cavity prior to closing the cavity. Note that the step of determining whether all the target tissue has been removed may be accomplished before or after expandable element 194 has been positioned within void 190.

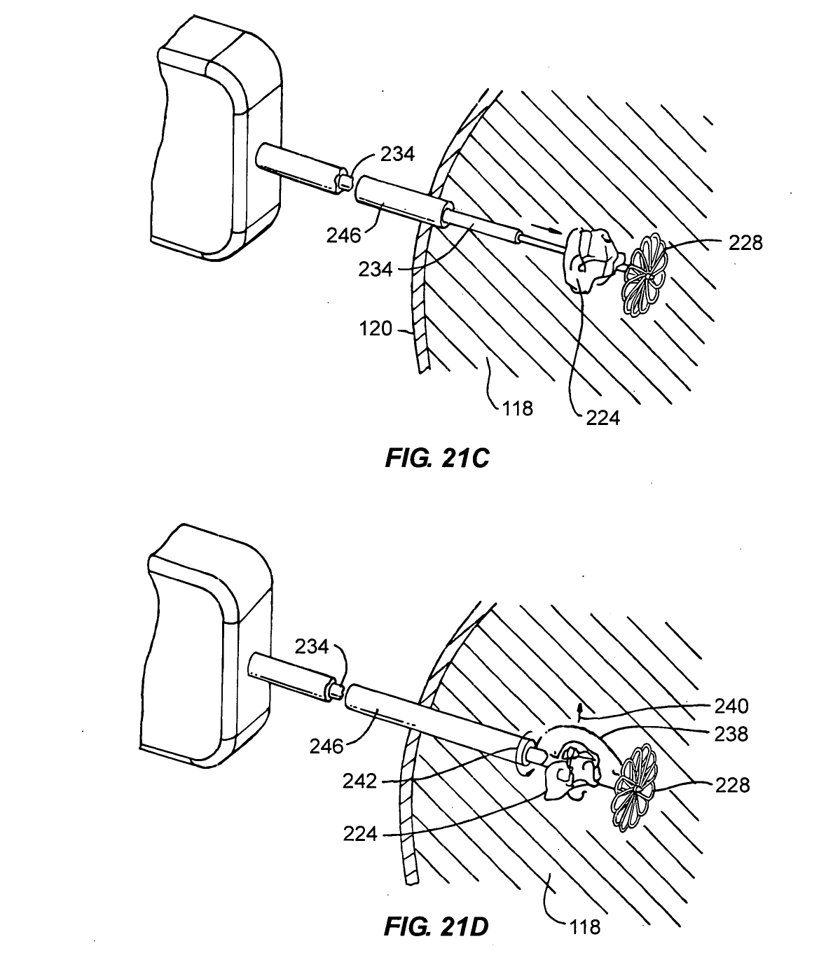

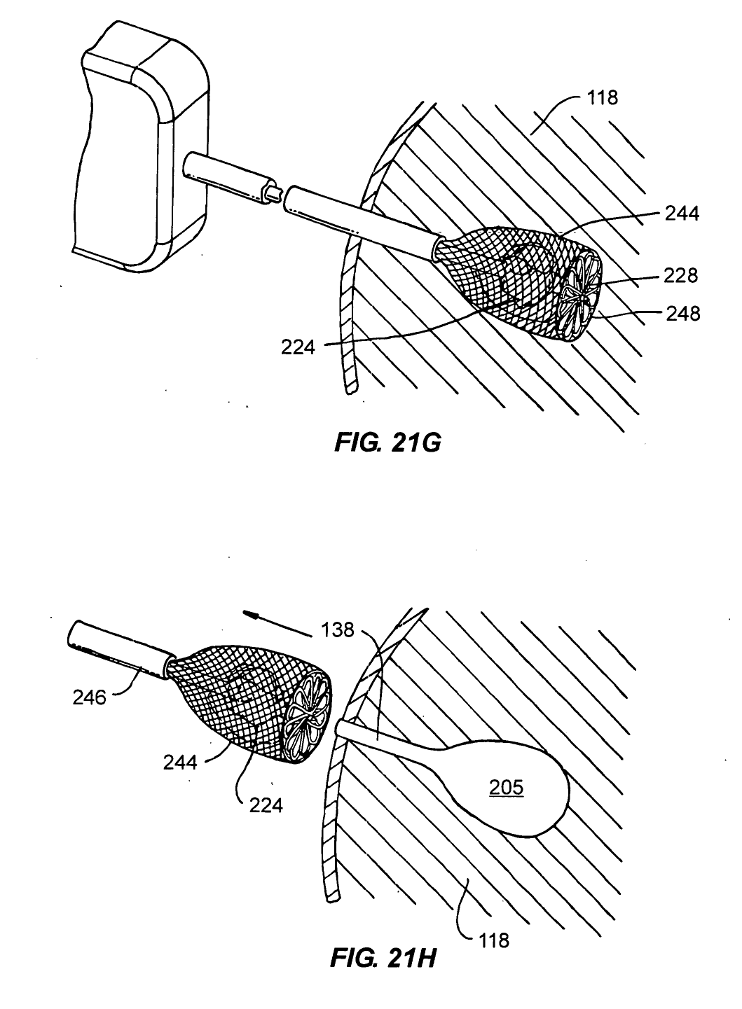

FIGS. 17C-17H show one method of separating tissue layer 200 from the surrounding tissue 118 by passage of a loop separator 202, shown also in FIGS. 18A-18F, over insertion device 192 and through sheath 105. Loop separator 202 includes a sheath 204 through which a cutter wire 206 passes. A loop 208 of wire 206 extends from the distal end 210 of sheath 204. As the distal end 210 of sheath 204 is moved distally, wire 206 is manipulated so that loop 208 first gets larger in size and then gets smaller in size as the loop passes around expanded balloon 194 thus separating tissue layer 200 from the surrounding tissue 118. To aid the cutting action of loop 208, the loop may, for example, have sharpened or roughened edges or the loop may be energized, such as by heating, or be supplied with mechanical vibrational or oscillatory energy. Other methods for separating tissue layer 200 may include, for example, the use of radially expandable and rotatable cutter wires as illustrated in FIGS. 11A-11C, the use of a mesh cutter as is discussed below with reference to FIGS. 19A-19D, or the use of tissue separation structure as is illustrated in FIGS. 21A-21H. After separating tissue layer 200 from the surrounding tissue 118, loop separator 202 may be removed for the subsequent removal of tissue layer 200 surrounding expanded element 194. FIG. 17F proposes the removal of tissue there 200 and expanded element 194 through introducer sheath 205 by the use of suction as indicated by arrow 211. FIG. 17G suggests the use of a mesh type capturing mechanism 213 to envelop tissue layer 200 for removal from the patient. Capturing mechanism 213 may be similar to the tubular mesh material 212 discussed below with regard to FIGS. 19A-19D. Other types of capturing mechanisms may be used as well. In addition, loop separator 202 may be left in place and removed with tissue layer 200 during an appropriate procedure.

FIG. 17I illustrates, in simplified form, a cross-sectional view of tissue layer 200 removed from the patient. Tissue layer 200 comprises an inner, void-defining surface 201 and an outer surface 203. Outer surface 203 may be tested to check for the presence of target tissue so to determine if all the target tissue has been removed. If outer surface 203 tests positive for the presence of diseased tissue, a determination must be made as to how to deal with the diseased tissue remaining within the patient and surrounding the enlarged void 205 shown in FIG. 17H. One procedure may be to repeat the procedure using an enlarged expandable element 194 sized to fit within enlarged void 205. Other surgical or non-surgical techniques may be used as well. If it is determined that all of the target tissue has been removed, then the patient is closed in the usual fashion. However, there may be a need for access for additional or adjunctive therapy. Even further, another material or an implant may be placed inside the cavity prior to closing the cavity.

FIG. 19A illustrates the situation shown in FIG. 17B, that is with expandable element 194 expanded at target site 126, with the use of a tubular, radially expandable mesh cutter 212 to separate tissue layer 200 from surrounding tissue 118. Mesh cutter 212 is typically made of an electrically conducting metal or other material that will sever the tissue mechanically. Mesh cutter 212 is constructed so that when placed in compression, the distal, cutting edge 214 tends to radially expand. This is suggested in FIG. 19A. The amount and rate of radial expansion of cutting edge 214 may be controlled by, for example, the use of a pull wire or loop along the cutting edge. As cutter 212 continues to move distally from between inner and outer tubes 215, 217, distal cutting edge 214 is gradually pulled down to the closed condition of FIG. 19C so that mesh cutter 212 completely envelops tissue layer 200 to permit tissue layer 200, together with expandable element 194 therein, to be withdrawn simultaneously with mesh cutter 212 as suggested in FIG. 19D. This procedure helps to ensure tissue layer 200 is substantially intact for examination by the physician or other health-care professional.

Another intraoperative treatment method, which may advantageously take place following the removal of target tissue from a target site leaving access, typically using sheath 105, to void 190 at the target site, relates to placing a flexible implant 216 into the void through the sheath. FIGS. 20A-20C illustrate the placement of a bag-type flexible implant 216, made of non-bioabsorbable material, through sheath 105 and into void 190 to at least substantially filling void. Implant 216 may also be a bioabsorbable material, such as collagen or a gel, that is eventually replaced with tissue. After flexible implant 216 is in place, sheath 105 may be removed as suggested in FIG. 20C. By maintaining sheath 105 in place after removal of tissue from the target site, the implant placement takes place in an efficient manner without the additional trauma and expense that would result if placed postoperatively. Other types of flexible implants, such as an implant that may be inflated once in place within the void, could be used. The flexible implant will typically be filled with a flowable, or at least a formable, material, such as a liquid, a gel, a granular material, or a combination thereof. Implant 216 preferably substantially fills void 190, that is fills at least about 60 percent of void 190, and may be sized to completely fill void 190 or to overfill, and thus enlarge, void 190, such as by about 20 percent or more.

A further intraoperative tissue treatment method using suction is disclosed in FIGS. 21A-21H. FIG. 21A illustrates a suction device 220 passing through skin surface 120. Device 220 has a tubular body 221 with suction inlets 222 at its distal end, the suction inlets positioned within void 190. Fluid, typically including liquid, gas and the occasional particles, is withdrawn through suction inlets 222 so to collapse tissue 118 surrounding void 190 to create collapsed tissue 224 at target site 126 as shown in FIG. 21B. Suction device 220 has, in this embodiment, a radially expandable blocking element 226 at the distal end of body 221. Blocking element 226, in this embodiment, comprises numerous individual wires 228 which can be directed out through openings 230 formed at the distal end of tubular body 221. Blocking element 226 is positioned distally of collapsed tissue 224 at target site 226. A tissue separator assembly 232, see FIGS. 21C-21E, includes a rotatable tube 234 which passes over shaft 221 until its distal end 236 extends between collapsed tissue 224 and blocking element 226. Once in position, a wire tissue cutter 238 extends radially outwardly as indicated by an arrow 240 of FIG. 21B; tube 234 is then rotated as indicated by arrow 242 so to cut a layer of tissue 200 surrounding target site 226. To help preserve the integrity of tissue layer 200 during and subsequent to the removal of the tissue layer from the patient, a radially expandable, tubular mesh material 244 is extended out from between an outer tube 246 and rotatable tube 234 of assembly 232. Mesh material 244 may be constructed similarly to the material described with regard to FIGS. 19A-19D so that it tends to expand radially outwardly when placed under compression. The outer edge 248 of mesh material 244 tends to follow the dissection plane between the outer surface 203 of tissue layer 200 and the surrounding tissue 118. Once in the position of FIG. 21G, with outer edge 248 adjacent to blocking element 226, assembly 232 and tissue layer 200 housed within mesh material 244 can be removed in unison as indicated in FIG. 11H with tissue layer 200 substantially intact for subsequent examination.

FIGS. 17H and 21H each show an enlarged void 205 and a relatively narrow tissue track 138. The tissue 118 is quite elastic and very often permits the removal of an enlarged mass along a relatively narrow tissue track, after which the elastic nature of the tissue tends to cause the tissue to return to its prestretched condition. If desired, a second, enlarged expandable element 194 may be placed in the enlarged void 205. If the outer surface 203 of tissue layer 200 is found to contain diseased tissue, a second excisional procedure as described above or some other therapeutic procedure, may be accomplished if considered necessary or desirable. If outer surface 203 is found not to contain diseased tissue, enlarged void 205 may have a hemostatic, bioabsorbable implant inserted into the void; in some situations it may be desired to place a flexible implant 216 into void 205, especially while sheath 205 is maintained in place.

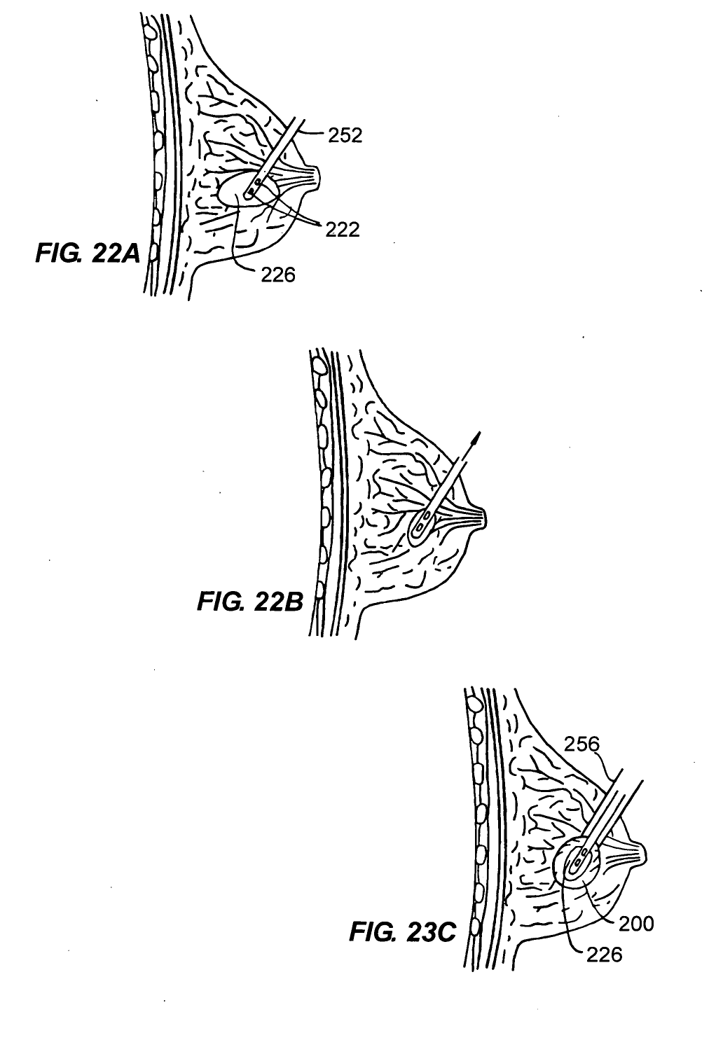

FIGS. 22A-22C show an alternative to the method of FIGS. 21A-21H. A suction device 252 extends along the tissue track and has suction inlets 222 at its distal end. After at least partially collapsing the tissue surrounding suction inlets 222, see FIG. 22B, a rotating blade tissue cutter 256 is used to create tissue layer 200 at target site 26. Removal of tissue layer 200 can be in a manner similar to that discussed above with regard to FIGS. 13A-16C and 17A-17H.

Modification and variation can be made to the disclosed embodiments of FIGS. 11A-22C. For example, blocking element 226 and/or mesh material 244, as well as other structure, may be used to remove tissue surrounding an expanded expandable element 194. The methods and devices of FIGS. 17A-19D may be used to remove collapsed tissue 224 of FIGS. 21B-21H. In some situations it may be necessary or desirable to temporarily enlarge tissue track 138, such as using the devices and methods of FIGS. 13A-16C.

The Occlusion, Anchoring, Tensioning and Flow Direction Apparatus

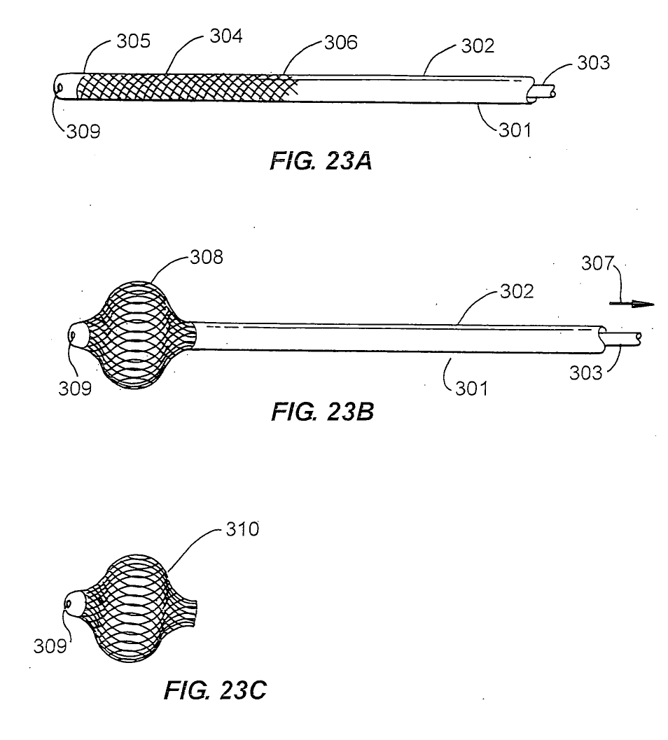

Although the instant invention of FIGS. 23-26 relates to four basic embodiments, those being flow directed, anchoring, tensioning and occluding, the instant invention is submitted for prosecution because the four embodiments are so closely related. Further and equally important is that the mechanical configuration(s) for all four embodiments of the present invention are similar.

The device of the instant invention is used for intervention into the tubular channels (lumens) of the body including, but not limited to arteries, veins, biliary tract, urological tract, intestines, nasal passages, ear canals, etc. Further, it can be useful as a suturing anchor in places of the body including, but not limited to adhering the stomach or other intestine to the abdominal wall in the case of feeding gastrostomies, jejunostomies, etc. Other anchoring applications of the instant invention include MIS facelifts and the repair of ptotic breasts. Even further, the instant invention is used for the repair of aneurysms of other permanent vessel occlusions. Such other permanent vessel occlusions would have applicability for occlusion of tributaries of vessels for vessel harvesting. The instant invention is particularly convenient to use in an operating room, interventional suite, patients' bedside, in an emergency room environment or in any emergency situation. One preferred embodiment of the instant invention is that it is inserted into the tubular channel of the body to utilize the flow directed characteristics of the invention. Once the device is in a flow/differential pressure situation, the inner core, mandrel/wire/string/member is deployed (usually pulled by the physician outside the body) so that the umbrella/trap configuration on the distal portion of the device opens. At the same time, the distal portion of the device becomes ‘floppy’ in nature so that it will follow the tortuous paths of the lumen without causing deleterious complications normally realized with conventional guide wires where they inadvertently damage the inner wall of the vessel when trying to cross said tortuous paths. The device is then carried in the direction of flow or of lower pressure (or with any contractile forces that may exist).

Once the device is in the desired position within the body, the umbrella like mechanism may or may not be un-deployed. In this case, once the device is removed from the package and before insertion into the body, the mechanism on the distal portion of the guide wire may be unopened (normally closed).

Alternatively, the device could have a distal configuration that causes it be moved in the direction of flow or in the direction of less pressure (or with the contractile forces) at the time it is opened from the package (e.g. normally opened). In this case the device is placed in the motion situation in the tubular channel of the body and is carried to the desired location. In the normally open position, the device may be very floppy in nature so that it will easily travel through the lumen of the body due to the pressure differential/flow/contractile forces. Once in position, the mechanism at the distal portion of the device may or may not be closed by some other mechanical means by the technician outside the body. One way of undeploying the distal ‘umbrella’ mechanism is by re-inserting the inner core so that the expanded mechanism becomes small or in its radially compressed state. Another advantage of re-inserting the inner core wire into the outer ‘floppy’ tube would be to make the support wire somewhat stiff, facilitating the insertion of another device over, through or along side the support wire that is attached to the expandable mechanism. Further, the umbrella like mechanism could become enlarged so that it will anchor in the lumen to keep its desired position.

Possible configurations of the distal mechanism are varied. One such mechanism is a balloon that is inflated for flow and deflated when not required. Another configuration that could be used is a mechanism known as a malecot. This malecot is a common configuration used in catheters for holding them in place (in the case of feeding tubes in the intestines). It is usually a polymeric tube that has four slits diametrically opposed. When the distal tip of the malecot is put into compression (usually by pulling an inner wire or member), the four sides of the polymer are pushed outward so as to create a larger diameter on the distal tip. Alternatively, the normal configuration of the malecot could be an open configuration whereby, when put into tension (large or small), the malecot closes to come near to or equal to the diameter of the elongated member. This larger diameter is larger than the body length of the catheter or wire. Another alternative is one that is similar to the malecot, but uses a multi-stranded braid on the distal end. When the braid is put into compression, the braid is pulled together and it flares out to create a larger diameter only the distal end. Alternatively either the braid or the malecot can have a permanent set put into in so that it is normally open or of the larger diameter. In this case, when it is put into tension (usually from some inner core wire or mandrel) it collapses down to the diameter of the body of the wire or catheter. Even further, the expandable mechanism on the distal end of these devices could be programmed to be thermally sensitive so that they expand or contract when placed in desired thermal gradients. One such mechanism for ‘programming’ materials like this is known as Shaped Memory Alloys (SMA) or Two Way Shaped Memory Alloys (TWSMA). Another exemplary embodiment of the instant invention is that once the device is placed in its desired location the mechanism (usually near the distal portion of the device) is deployed to ‘lock’ or ‘anchor’ it in the desired position.

Another embodiment is the tensioning characteristic of the instant invention. When the device is in or near a desired location of the body, the distal mechanism is deployed so that it anchors or has a tendency not to move. In this configuration, the wire, catheter or other device can be put into tension that will allow the passage of another device over or with the inner support wire. Even further and discussed heretofore, the instant invention can be ‘detached’ from the support wire and act as a tubular channel occluder.

This anchoring mechanism may or may not be used with the other embodiments. Further, the flow/contractile force characteristic may or may not be used with the other embodiments. Even further, the tensioning characteristic may or may not be used with the other embodiments. Last, the occluder may be used independently of the other three. In other words, although the distal mechanism that is used for all four embodiments may be similar to one another, the separate four embodiments may be used alone or in combination with the other embodiments.

Referring now to the figures, the four embodiments of the instant invention are illustrated.

Turning now to FIG. 23-A, a preferred embodiment of the instant invention is illustrated using a schematic drawing. The radially compressed, smaller support wire 301 is illustrated. The shaft 302 is a tubular outer shell where the inner wire or tube 303 rests. The inner tube 303 is attached to the distal end of the annular braid 304 at 305. The outer shell 302 may be attached to the annular braid at 306. In the case of the detachable occluder in FIG. 23-C, it may not be attached so that the occluder is set free in the desired location.

Referring now to FIG. 23-B, the inner tube or wire 303 is moved relative to the outer shell 302 as indicated by the arrow 307. This relative motion causes the annular braid 304 to expand radially as shown at 308. The shapes shown in these figures show an ovoid shape, however the shape can vary significantly as described heretofore. Notice that there is a through lumen illustrated inside the inner tube 303 and is further indicated at the distal tip of the assembly by 309. This may or may not be required depending on the application.

Turning now to FIG. 23-C, the preferred embodiment of the instant invention as an occluder 310 is illustrated in the schematic.

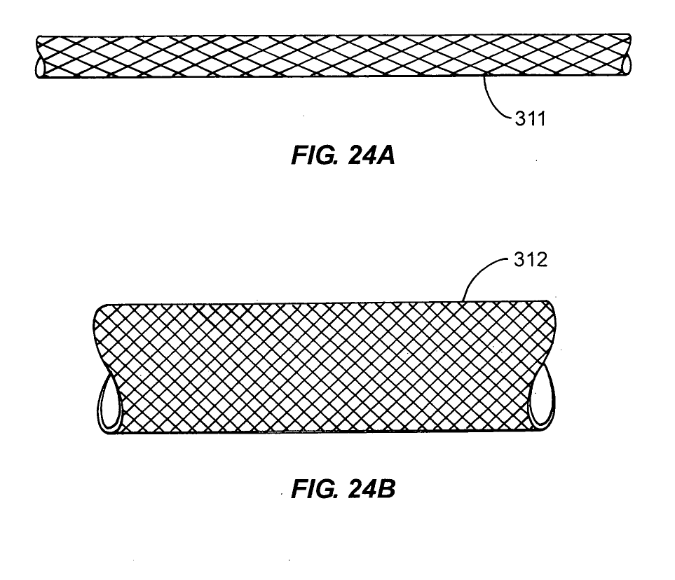

Referring now to FIGS. 24-A and 24-B, a schematic view of the annular or tubular braid is illustrated. FIG. 24-A illustrates the annular braid in its relaxed, smaller or compressed state 311. FIG. 24-B illustrates the annular braid in its expanded state 312. The expansion is achieved by putting the braid into a compressive mode and changing the overall length of the braid. This can also be accomplished with self expanding of the braid by programming it with thermal treatments or using SMA (Shaped Memory Alloys) or by using a thermal change to change the shape of the device with a technique known as TWSMA (Two Way Shape Memory Alloy).

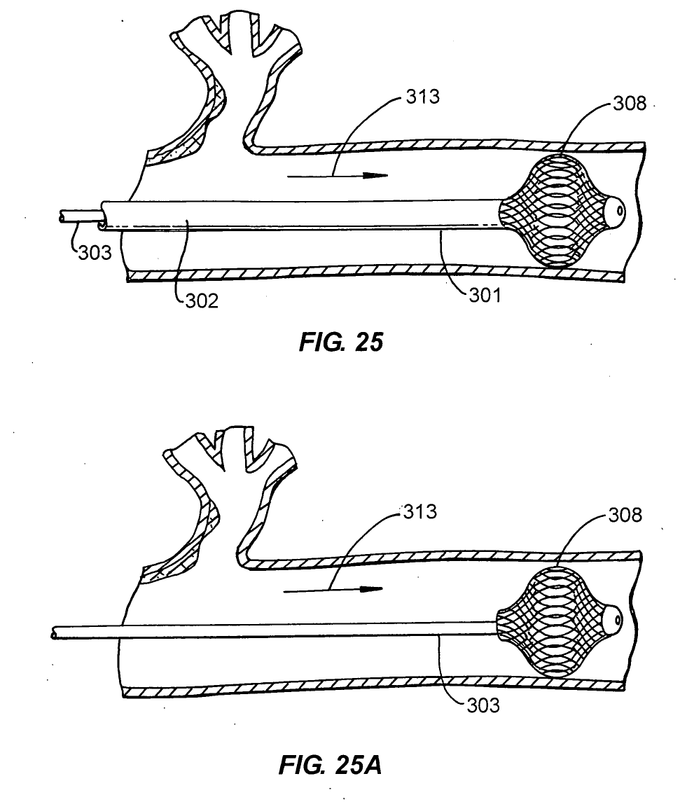

Turning now to FIG. 25, a preferred embodiment is illustrated in a schematic view. This is the expanded device 301 in place in a tubular channel of the body. This figure shows the instant invention as anchor and subsequent tensioner if so desired for the particular application. Further, it could be the preferred embodiment of a flow directed guide wire or device if there is flow in the tubular channel as indicated by the arrow 313. The mechanisms of the preferred embodiment are shown here in FIGS. 25 & 26 inside a tubular channel. However, the preferred embodiment of the instant invention could be used for other anchoring as heretofore disclosed. This anchor could be used for closing percutaneous punctures in the femoral artery for example as well. This is a ubiquitous problem. By deploying the anchor on the inside of the puncture of the vessel (artery or vein), the puncture wound would seal faster. Further dehydrated collagen could be used to aid in this procedure. Even further, this anchor or occluder could be fabricated with bio-resorbable materials as required for the particular application.

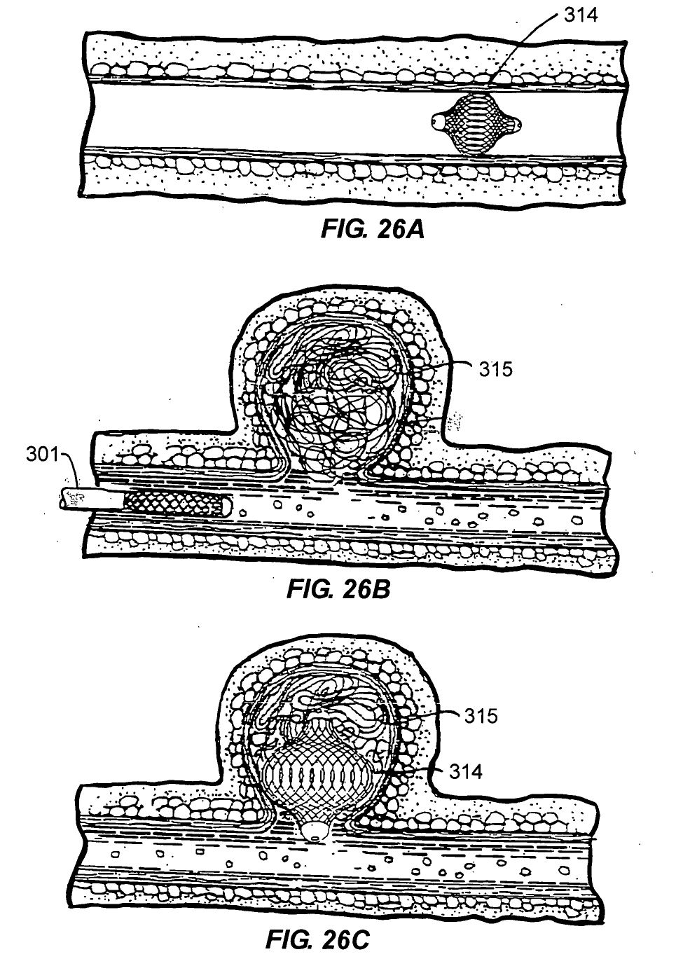

Turning now to FIG. 26-A, a schematic illustration shows the occluder 314 in place in the vessel. This is accomplished by removing the support wire (inner wire or tube 303 and outer shell 302) as described heretofore. FIG. 26-B shows the instant invention 301 in its smaller condition as it is being passed into a vessel with an aneurysm 315. FIG. 26-C illustrates the occluder 314 in position in the aneurysm thus providing a novel therapy to this dangerous disease.

In any of these instances, the ‘desired’ location of the device is usually determined using Image Intensification (Fluoroscopy, Ultrasound Imaging, MRI, etc.). Further, the location could be monitored using cameras or other visualization techniques.

The Tubular Braid Elements

The apparatus of the instant invention includes an elongate tube; an elongate mandril inside the tube and an expandable tubular braid. The elongate mandril extends from the proximal end of the device to the distal end. The elongate tube usually extends from close to the proximal end of the device to close to the distal end. The distal end of the tubular braid is bonded to the distal end of the inner elongate mandril. The mandril may extend beyond the tubular braid. The proximal end of the tubular braid is bonded to the distal end of the elongate tube.

The braid may be open, but may be laminated or covered with a coating of elastic, generally inelastic, plastic or plastically deformable material, such as silicone rubber, latex, polyethylene, thermoplastic elastomers (such as C-Flex, commercially available from Consolidated Polymer Technology), polyurethane and the like. The assembly of tube, mandril and braid is introduced percutaneously in its radially compressed state. In this state, the outside diameter of the braid is close to the outside diameter of the elongate tube. This diameter is in the range of 10 to 500 mils, and usually 25 to 250 mils (i.e. thousandth of an inch). After insertion, moving the mandril proximally with respect to the tube expands the tubular braid.

The tubular braid is preferably formed as a mesh of individual non-elastic filaments (called “yarns” in the braiding industry). However, it can have some elastic filaments interwoven to create certain characteristics. The non-elastic yarns can be materials such as polyester, PET, polypropylene, polyamide fiber (Kevlar, DuPont), composite filament wound polymer, extruded polymer tubing (such as Nylon II or Ultem, commercially available from General Electric), stainless steel, Nickel Titanium (Nitinol), or the like so that axial shortening causes radial expansion of the braid. These materials have sufficient strength so that the expanding element will retain its expanded condition in the lumen of the body while removing the matter therefrom. Further, all expandable mechanisms described heretofore, can be manufactured using shape memory materials so that they are self expanding or even expandable when certain temperatures or thermal energies are delivered to the mechanisms. Such material characteristics can be accomplished with different programming methods such as, but not limited to Two Way Shape Memory (TWSM) alloys.

The braid may be of conventional construction, comprising round filaments, flat or ribbon filaments, square filaments, or the like. Non-round filaments may be advantageous to decrease the axial force required for expansion to create a preferred surface area configuration or to decrease the wall thickness of the tubular braid. The filament width or diameter will typically be from about 0.5 to 50 mils, usually being from about 5 to 20 mils. Suitable braids are commercially available from a variety of commercial suppliers.

The tubular braids are typically formed by a “Maypole” dance of yarn carriers. The braid consists of two systems of yarns alternately passing over and under each other causing a zigzag pattern on the surface. One system of yarns moves helically clockwise with respect to the fabric axis while the other moves helically counter-clockwise. The resulting fabric is a tubular braid. Common applications of tubular braids are lacings, electrical cable covers (i.e. insulation and shielding), “Chinese hand-cuffs” and reinforcements for composites. To form a balanced, torque-free fabric (tubular braid), the structure must contain the same number of yarns in each helical direction. The tubular braid may also be pressed flat to form a double thickness fabric strip. The braid weave used in the tubular braid of the present invention will preferably be of the construction known as “two dimensional, tubular, diamond braid” that has a 1/1 intersection pattern of the yarns which is referred to as the “intersection repeat”. Alternatively, a Regular braid with a 2/2 intersection repeat and a Hercules braid with an intersection repeat of 3/3 may be used. In all instances, the helix angle (that being the angle between the axis of the tubular braid and the yarn) will increase as the braid is expanded. Even further, Longitudinal Lay-Ins can be added within the braid yarns and parallel to the axis to aid with stability, improve tensile and compressive properties and modulus of the fabric. When these longitudinal “Lay-In” yarns are elastic in nature, the tubular braid is known as an elastic braid. When the longitudinal yarns are stiff, the fabric is called a rigid braid. Biaxially braided fabrics such as those of the present invention are not dimensionally stable. This is why the braid can be placed into an expanded state from a relaxed state (in the case of putting it into the compressive mode). Alternatively this could be a decreased/reduced (braid diameter decreases) state when put into tension from the relaxed state. When put into tension (or compression for that matter) the braid eventually reaches a state wherein the diameter will decrease no more. This is called the “Jammed State”. On a stress strain curve, this corresponds to increase modulus. Much of the engineering analyses concerning braids are calculated using the “Jammed State” of the structure/braid. These calculations help one skilled in the art to design a braid with particular desired characteristics. Further, material characteristics are tensile strength, stiffness and Young's modulus. In most instances, varying the material characteristics will vary the force with which the expanded condition of the tubular can exert radially. Even further, the friction between the individual yarns has an effect on the force required to compress and un-compress the tubular braid. For the present invention, friction should be relatively low for a chosen yarn so that the user will have little trouble deploying the engaging element. This is particularly important when the engaging element is located a significant distance from the user. Such is the case when the percutaneous entry is the groin (Femoral Artery for vascular interventions) and the point of engaging the engaging element is some distance away (i.e. the Carotid Artery in the neck). Similarly, this is true for long distances that are not vascular or percutaneous applications.

An exemplary device has the following characteristics:

a. Working Length:

i. 30-500 cm

b. Working Diameter:

i. The guide wire, catheter, endoscope or other device of the present idea has an outer diameter that ranges from 0.006″ to 0.315″, but can extend to smaller and larger sizes as technology and procedures require.

c. Physical Configuration:

i. The device of the present idea will have a predetermined shaped (probably circular in diameter of 6-10″) coiled in the package, “as supplied”. Alternatively the product/device may be supplied straight but may have a shape at the distal end. The distal end may be tapered to a smaller distal diameter. This tapering may occur in the distal 6-12″ of the device, but could occur over a greater length and there may be more than one taper along its length. Optionally, the device may have a shaped tip or a tip that may be malleable so that the user prior to introduction may shape it.

The device of the instant invention may have conventional lubricious coatings to enhance introduction into the target body lumen, e.g. hyaluronic or other equivalent coatings. Further, the user, prior to insertion may apply a lubricious coating. This may be extremely useful in the case of a reusable device (like an endoscope). As an advantage of the present idea, the device will be less difficult to feed it to the desired location in the body. Further difficulty will be greatly decreased for placement of other devices over or with the inner device. Even further, the instant invention will be less difficult to remain in the target location. This decreased difficulty will decrease cost due to time in the Operating Room (Operating Rooms costs are estimated in excess of $90 dollars per minute in the U.S.) or other environment. Additionally, the decrease in difficulty will aid in patient care and the potential in deleterious effects due to the inability to place the device in the appropriate position in the patient and keep it there or to place other devices with the present idea.

An exemplary device having an expanding ‘umbrella’ mechanism located on its distal tip is illustrated in the figures. This mechanism may be at the tip or somewhere else in the distal portion of the device. Additionally, this mechanism may be any of a number of mechanisms that will help aid in moving the device using the physiological environment of the body. Alternatively, this distal mechanism may be used for anchoring, flow direction, tensioning or occluding. In this particular embodiment, a distal portion of the device may not coiled and will thus retain the malleable or resilient characteristics typical of conventional devices.

Although the foregoing idea has been described in some detail by way of illustration and example, for purposes of clarity of understanding, it will be obvious that certain changes and modifications may be practiced within the scope of the appended claims. The disclosures of any and all patents, patent applications and printed publications referred to above are hereby incorporated by reference.

1. A medical device for the use in diagnosis and/or treatment of cardiovascular disease in the human body comprising:
a catheter having a proximal catheter end and a distal catheter end and defining a lumen extending from the distal catheter end towards the proximal catheter end, the catheter adapted for use in diagnosis and/or treatment of cardiovascular disease in the human body;
a first expandable and contractible, vessel-occluding element positioned distal of the distal catheter end;
a second expandable and contractible, annular-space-blocking element positioned between the first expandable and contractible element and the proximal catheter end; and
at least one of the first and second expandable and contractible elements comprising spaced apart structural members and a membrane associated therewith.

2. The medical device according to claim 1 wherein the second expandable and contractible element is positioned at and extends from the catheter distal end.

3. The medical device according to claim 1 wherein the second expandable and contractible element comprises a multiple wing, malecot type of expandable and contractible element.

4. The medical device according to claim 1 wherein the second expandable and contractible element comprises a membrane.

5. The medical device according to claim 1 wherein the second expandable and contractible element comprises a multiple wing, malecot type of expandable and contractible element and a membrane associated therewith.

6. The medical device according to claim 5 wherein the membrane covers the multiple wing, malecot type of expandable and contractible element.

7. The medical device according to claim 1 wherein the first expandable and contractible element comprises a braided element.

8. The medical device according to claim 1 wherein the first expandable and contractible element comprises spaced apart structural members.

9. The medical device according to claim 1 wherein the first expandable and contractible element comprises spaced apart structural members and a membrane associated therewith.

10. The medical device according to claim 1 wherein the second expandable and contractible element comprises spaced apart structural members.

11. The medical device according to claim 1 wherein the second expandable and contractible element comprises spaced apart structural members and a membrane associated therewith.

12. The medical device according to claim 1 wherein the first and second expandable and contractible elements comprises spaced apart structural members.

13. The medical device according to claim 1 wherein at least one of the first and second expandable and contractible elements comprises spaced apart structural members and a membrane associated therewith.

14 The medical device according to claim 1 wherein the first and second expandable and contractible elements comprises spaced apart structural members and a membrane associated therewith.

15. The medical device according to claim 1 wherein the first expandable and contractible element comprises a braided element covered with a membrane.

16. The medical device according to claim 1 wherein the first expandable and contractible element comprises a native vessel sealing element.

17. The medical device according to claim 1 wherein a chosen one of the first and second expandable and contractible elements is funnel-shaped when in an expanded state.

18. The medical device according to claim 1 wherein a chosen one of the first and second expandable and contractible elements has a longitudinally-extending opening to permit material to pass therethrough.

19. The medical device according to claim 1 wherein the first expandable and contractible element is movable relative to the second expandable and contractible element.

20. The medical device according to claim 1 wherein the membrane is impermeable.

21. The medical device according to claim 1 wherein the membrane is elastomeric.

22. A medical device for the use in diagnosis and/or treatment of cardiovascular disease in the human body comprising:
a catheter having a proximal catheter end and a distal catheter end and defining a lumen extending from the distal catheter end towards the proximal catheter end, the catheter adapted for use in diagnosis and/or treatment of cardiovascular disease in the human body;
a first expandable and contractible, vessel-occluding element positioned distal of the distal catheter end;
a second expandable and contractible, annular-space-blocking element positioned between the first expandable and contractible element and the proximal catheter end; and
a chosen one of the first and second expandable and contractible elements being having a funnel-shaped surface, when in an expanded state, and having a longitudinally-extending opening to permit material to pass therethrough for receipt of material.

23. The medical device according to claim 22 wherein the second expandable and contractible element is positioned at and extends from the catheter distal end.

24. The medical device according to claim 22 wherein the second expandable and contractible element comprises a multiple wing, malecot type of expandable and contractible element.

25. The medical device according to claim 22 wherein the second expandable and contractible element comprises a membrane.

26. The medical device according to claim 22 wherein the second expandable and contractible element comprises a multiple wing, malecot type of expandable and contractible element and a membrane associated therewith.

27. The medical device according to claim 26 wherein the membrane covers the multiple wing, malecot type of expandable and contractible element.

28. The medical device according to claim 22 wherein the first expandable and contractible element comprises a braided element.

29. The medical device according to claim 22 wherein the first expandable and contractible element comprises spaced apart structural members.

30. The medical device according to claim 22 wherein the first expandable and contractible element comprises spaced apart structural members and a membrane associated therewith.

31. The medical device according to claim 22 wherein the second expandable and contractible element comprises spaced apart structural members.

32. The medical device according to claim 22 wherein the second expandable and contractible element comprises spaced apart structural members and a membrane associated therewith.

33. The medical device according to claim 22 wherein at least one of the first and second expandable and contractible elements comprises spaced apart structural members.

34. The medical device according to claim 22 wherein the first and second expandable and contractible elements comprises spaced apart structural members.

35. The medical device according to claim 22 wherein at least one of the first and second expandable and contractible elements comprises spaced apart structural members and a membrane associated therewith.

36. The medical device according to claim 22 wherein the first and second expandable and contractible elements comprises spaced apart structural members and a membrane associated therewith.

37. The medical device according to claim 22 wherein the first expandable and contractible element comprises a braided element covered with a membrane.

38. The medical device according to claim 22 wherein the first expandable and contractible element comprises a native vessel sealing element.

39. The medical device according to claim 22 wherein the first expandable and contractible element is movable relative to the second expandable and contractible element.

40. The medical device according to claim 22 wherein at least one of the first and second expandable and contractible elements comprises a balloon.

41. A medical device for the use in diagnosis and/or treatment of cardiovascular disease in the human body comprising:
a catheter having a proximal catheter end and a distal catheter end and defining a lumen extending from the distal catheter end towards the proximal catheter end, the catheter adapted for use in diagnosis and/or treatment of cardiovascular disease in the human body;
a support element extending distally of the distal catheter end;
a first expandable and contractible, vessel-occluding element mounted to the support element and positioned distal of the distal catheter end;
a second expandable and contractible, annular-space-blocking element mounted to the catheter and positioned between the first expandable and contractible element and the proximal catheter end;
a chosen one of the first and second expandable and contractible elements being having a funnel-shaped surface, when in an expanded state, and having a longitudinally-extending opening to permit material to pass therethrough for receipt of material; and
at least one of the first and second expandable and contractible elements comprising spaced apart structural members and a membrane associated therewith.

42. The medical device according to claim 41 wherein a portion of the support element is housed within the catheter.

43. The medical device according to claim 41 wherein a portion of the support element is slidably housed within the catheter.

44. The medical device according to claim 41 wherein the first expandable and contractible element comprises a braided element.

45. The medical device according to claim 41 wherein the first expandable and contractible element comprises a braided element covered with a membrane.

46. The medical device according to claim 41 wherein the first expandable and contractible element comprises a native vessel sealing element.

47. A medical device for the use in diagnosis and/or treatment of cardiovascular disease in the human body comprising:
a catheter having a proximal catheter end and a distal catheter end and defining a lumen extending from the distal catheter end towards the proximal catheter end, the catheter adapted for use in diagnosis and/or treatment of cardiovascular disease in the human body;
a first expandable and contractible, vessel-occluding element positioned distal of the distal catheter end; and
a second expandable and contractible, annular-space-blocking device-occluding element positioned between the first expandable and contractible element and the proximal catheter end.

48. The medical device according to claim 47 wherein the second expandable and contractible element is positioned at and extends from the catheter distal end.

49. The medical device according to claim 47 wherein the second expandable and contractible element comprises a multiple wing, malecot type of expandable and contractible element.

50. The medical device according to claim 47 wherein the second expandable and contractible element comprises a membrane.

51. The medical device according to claim 47 wherein the second expandable and contractible element comprises a multiple wing, malecot type of expandable and contractible element and a membrane associated therewith.

52. The medical device according to claim 51 wherein the membrane covers the multiple wing, malecot type of expandable and contractible element.

53. The medical device according to claim 47 wherein the first expandable and contractible element comprises a braided element.

54. The medical device according to claim 47 wherein the first expandable and contractible element comprises spaced apart structural members.

55. The medical device according to claim 47 wherein the first expandable and contractible element comprises spaced apart structural members and a membrane associated therewith.

56. The medical device according to claim 47 wherein the second expandable and contractible element comprises spaced apart structural members.

57. The medical device according to claim 47 wherein the second expandable and contractible element comprises spaced apart structural members and a membrane associated therewith.

58. The medical device according to claim 47 wherein at least one of the first and second expandable and contractible elements comprises spaced apart structural members.

59. The medical device according to claim 47 wherein the first and second expandable and contractible elements comprises spaced apart structural members.

60. The medical device according to claim 47 wherein at least one of the first and second expandable and contractible elements comprises spaced apart structural members and a membrane associated therewith.

61. The medical device according to claim 47 wherein the first and second expandable and contractible elements comprises spaced apart structural members and a membrane associated therewith.

62. The medical device according to claim 47 wherein the first expandable and contractible element comprises a braided element covered with a membrane.

63. The medical device according to claim 47 wherein the first expandable and contractible element comprises a native vessel sealing element.

64. The medical device according to claim 47 wherein a chosen one of the first and second expandable and contractible elements is funnel-shaped when in an expanded state.

65. The medical device according to claim 47 wherein a chosen one of the first and second expandable and contractible elements has a longitudinally-extending opening to permit material to pass therethrough.

66. The medical device according to claim 47 wherein the first expandable and contractible element is movable relative to the second expandable and contractible element.

67. The medical device according to claim 47 wherein the second expandable and contractible, device-occluding element comprises an artificial vessel-occluding element.

68. The medical device according to claim 47 wherein at least one of the first and second expandable and contractible elements comprises a balloon.

69. A medical device for the use in diagnosis and/or treatment of cardiovascular disease in the human body comprising:
a catheter having a proximal catheter end and a distal catheter end and defining a lumen extending from the distal catheter end towards the proximal catheter end, the catheter adapted for use in diagnosis and/or treatment of cardiovascular disease in the human body;
an expandable and contractible, annular-space-blocking element carried by the catheter at or near the distal catheter end;
the expandable and contractible element having a funnel-shaped surface, when in an expanded state, for receipt of material; and
the expandable and contractible element comprising spaced apart structural members and a membrane associated therewith.

70. The medical device according to claim 69 wherein the membrane is an impermeable membrane.

71. The medical device according to claim 69 wherein the membrane is elastomeric.

72. The medical device according to claim 69 wherein the expandable and contractible element comprises a braided element.

73. The medical device according to claim 69 wherein the expandable and contractible element comprises a braided element covered with the membrane.

74. The medical device according to claim 69 wherein the expandable and contractible element comprises a native vessel sealing element.
